(19) United States
(12) Patent Application Publication
(10) Pub. No.: US 2018/0064726 A1
(43) Pub. Date: Mar. 8, 2018

(54) Deuterium-Substituted Oxazepin Compounds

(71) Applicant: Auspex Pharmaceuticals, Inc., Frazer, PA (US)

(72) Inventors: Richard FISHER, Beit Shemesh (IL); Chengzhi ZHANG, San Diego, CA (US)

(21) Appl. No.: 15/697,950

(22) Filed: Sep. 7, 2017

Related U.S. Application Data
(60) Provisional application No. 62/384,776, filed on Sep. 8, 2016.

(51) Int. Cl. A61K 31/553; A61K 31/506; C07B 59/00; C01B 4/00

(52) U.S. Cl. CPC A61K 31/553; A61K 31/506; C07B 2200/05; C01B 4/00; A61K 2300/00; C07B 59/002

(57) ABSTRACT

Described are deuterium-substituted oxazepin compounds of structural Formula I, which are inhibitors/blockers of the late sodium current. Also described are pharmaceutical compositions comprising the deuterium-substituted oxazepin compounds, and methods of use thereof.

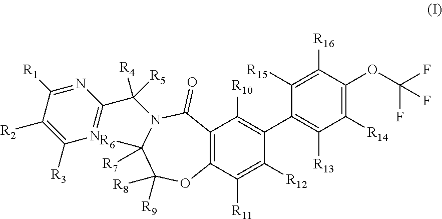

### CROSS-REFERENCE TO RELATED APPLICATIONS

This application claims the benefit of priority of U.S. provisional Application No. 62/384,776, filed Sep. 8, 2016, the disclosure of which is hereby incorporated by reference, as if written herein, in its entirety.

### TECHNICAL FIELD

The present invention relates generally to the field of pharmaceuticals and to methods of treating disorders. More particularly, provided herein are novel compounds which are inhibitors of the late sodium current and are useful in treating or preventing disorders including cardiovascular diseases and diabetes.

### BACKGROUND

The late sodium current (INaL) is a component of the fast Na⁺ current of cardiac myocytes and neurons. Late sodium current in cardiac cells is small compared with the fast component, but it may make a large contribution to sodium loading during each cardiac cycle. Impaired sodium channel function contributes to pathologic increase of the late sodium current, sodium overload, and sodium-induced calcium overload by way of the sodium-calcium exchanger. Calcium overload causes impaired diastolic relaxation, which increases diastolic wall tension, increases myocardial oxygen demand, reduces myocardial blood flow and oxygen supply, microvascular perfusion, and worsens ischemia and angina. Many common neurological and cardiac conditions are associated with abnormal (INaL) augmentation, which contributes to the pathogenesis of both electrical and contractile dysfunction in mammals. Inhibiting the late sodium current can lead to reductions in elevated intracellular calcium levels, which, in turn, may lead to reduced tension in the heart wall and reduced oxygen requirements for the heart muscle. Inhibition of cardiac late sodium current is a strategy used to suppress arrhythmias and sodium-dependent calcium overload associated with myocardial ischemia and heart failures. Thus, compounds that selectively inhibit the late sodium current (INaL) in mammals may be useful in treating such disease states.

Eleclazine (4-(pyrimidin-2-ylmethyl)-7-(4-(trifluoromethoxy)phenyl)-3,4-dihydrobenzo[b]oxepin-5(2H)-one]; CAS #1443211-72-0) is an inhibitor of the late sodium current. Eleclazine is being investigated for the treatment of cardiomyopathy, specifically hypertrophic cardiomyopathy, as well as additional cardiovascular indications, including angina, heart failure, atrial fibrillation (AF), ischaemic heart disorders, atrial premature beats (APBs), myocardial ischemia, and arrhythmias.

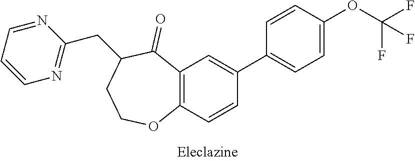 [SMILES: O=C1c2cc(-c3ccc(OC(F)(F)F)cc3)ccc2OCCC1Cc1ncccn1]

Eleclazine shows a shortening of the QTc interval (the time interval between the start of the Q-wave and the end of T-wave in the electrical cycle of the heart) in patients with QT-3 (LQT3) syndrome. LQTS is a genetic disorder that prolongs the heart's QTc interval and can cause life-threatening cardiac arrhythmias. Therefore, eleclazine is also being investigated for treatment of long QT syndrome.

Eleclazine may be metabolized in the liver and may be subject to extensive cytochrome P₄₅₀-mediated oxidative metabolism. Eleclazine is metabolized predominantly by N-dealkylation, and elimination is principally in the bile and gastrointestinal tract. The primary metabolite of eleclazine is GS-623134

Adverse effects associated with eleclazine may include dizziness, dry mouth, nausea, weakness, ringing in ears, tremors, and the like. Additionally, some metabolites of eleclazine, particularly the metabolite GS 623134, may have undesirable side effects.

Accordingly, there is a need for ion modulators, particularly late sodium current inhibitors, with improved pharmacokinetic properties.

### SUMMARY

Provided are deuterium-substituted oxazepin compounds, which are inhibitors of the late sodium current. Also provided are pharmaceutical compositions comprising the deuterium-substituted oxazepin compounds, and methods of use thereof, including methods for treatment or prevention of late sodium current-mediated disorders by administering, to a patient, the deuterium-substituted oxazepin compounds or pharmaceutical compositions comprising the deuterium-substituted oxazepin compounds. Further provided are methods of synthesizing the deuterium-substituted oxazepin compounds.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Eleclazine is a late sodium current inhibitor. The carbon-hydrogen bonds of eleclazine contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of such eleclazine in comparison with the compound having naturally occurring levels of deuterium.

Based on discoveries made in our laboratory, as well as considering the literature, eleclazine is likely metabolized in humans by oxidation of hydrocarbons and N-dealkylations. The approach described herein has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a pharmaceutical with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of eleclazine and attenuate interpatient variability.

Deuterium-substituted oxazepin compounds and pharmaceutical compositions employing such compounds, certain of which have been found to inhibit late sodium current activity have been discovered, together with methods of synthesizing and using the compounds, including methods for the treatment or prevention of late sodium current-mediated disorders in a mammal by administering the compounds as disclosed herein.

In certain embodiments of the present invention, the deuterium-substituted oxazepin compounds have a structure corresponding to Formula I:

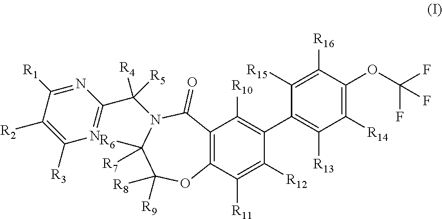

or a pharmaceutically acceptable salt, ester, prodrug, or solvate thereof, wherein:

R₁-R₁₆ are independently selected from hydrogen and deuterium, and at least one of R₁-R₁₆ is deuterium, with the proviso that when all of R₆, R₇, R₈, and R₉ are deuterium, at least one of R₁-R₅ or R₁₀-R₁₆ are also deuterium.

In further embodiments, said pharmaceutically acceptable salt is selected from a hydrochloride, a hydrobromide, a sulfate, a formate, an acetate, a fumarate, a citrate, a tartrate, a mesylate, a tosylate, a besylate, and the like.

In specific embodiments, said pharmaceutically acceptable salt is a hydrochloride salt.

In certain embodiments, R₁ is deuterium.

In certain embodiments, R₂ is deuterium.

In certain embodiments, R₃ is deuterium.

In certain embodiments, R₄ is deuterium.

In certain embodiments, R₅ is deuterium.

In certain embodiments, R₆ is deuterium.

In certain embodiments, R₇ is deuterium.

In certain embodiments, R₈ is deuterium.

In certain embodiments, R₉ is deuterium.

In certain embodiments, R₁₀ is deuterium.

In certain embodiments, R₁₁ is deuterium.

In certain embodiments, R₁₂ is deuterium.

In certain embodiments, R₁₃ is deuterium.

In certain embodiments, R₁₄ is deuterium.

In certain embodiments, R₁₅ is deuterium.

In certain embodiments, R₁₆ is deuterium.

In certain embodiments, R₄ and R₅ are deuterium.

In certain embodiments, R₄-R₅ and R₆-R₇ are deuterium.

In certain embodiments R₄-R₅ and R₈-R₉ are deuterium.

In certain embodiments R₄-R₅, R₆-R₇, and R₈-R₉ are deuterium.

Also provided herein are embodiments according to each of the embodiments above, wherein R₁-R₃ are deuterium.

Also provided herein are embodiments according to each of the embodiments above, wherein R₁₀-R₁₂ are deuterium.

Also provided herein are embodiments according to each of the embodiments above, wherein R₁₃-R₁₆ are deuterium.

Also provided herein are embodiments according to each of the embodiments above, wherein R₆-R₇ are hydrogen.

Also provided herein are embodiments according to each of the embodiments above, wherein R₈-R₉ are hydrogen.

Also provided herein are embodiments according to each of the embodiments above, wherein all of R₆-R₉ are hydrogen.

Also provided herein are embodiments according to each of the embodiments above, wherein every other substituent among R₁-R₁₆ not specified as deuterium is hydrogen.

In certain embodiments are provided compounds as disclosed herein, wherein at least one of R₁-R₁₆ independently has deuterium enrichment of no less than about 1%. In certain embodiments are provided compounds as disclosed herein, wherein at least one of R₁-R₁₆ independently has deuterium enrichment of no less than about 10%. In certain embodiments are provided compounds as disclosed herein, wherein at least one of R₁-R₁₆ independently has deuterium enrichment of no less than about 50%, including about 55%, about 60%, about 65%, about 70%, about 75%, about 80%, about 85%, about 90%, about 95%, and about 100%. In certain embodiments are provided compounds as disclosed herein, wherein at least one of R₁-R₁₆ independently has deuterium enrichment of no less than about 90%, including about 92%, about 94%, about 96%, about 98%, and about 100%. In certain embodiments are provided compounds as disclosed herein, wherein at least one of R₁-R₁₆ independently has deuterium enrichment of no less than about 95%, including about 96%, about 97%, about 98%, about 99%, and about 100%. In certain embodiments are provided compounds as disclosed herein, wherein at least one of R₁-R₁₆ independently has deuterium enrichment of no less than about 98%, including about 99%, and about 100%.

In further embodiments, the deuterium-substituted deuterium-substituted oxazepin compounds have a structure corresponding to Formula Ia

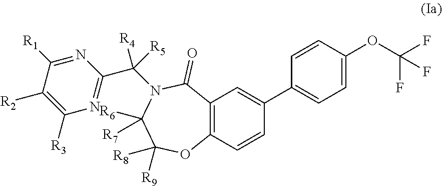

or a pharmaceutically acceptable salt, ester, prodrug, or solvate thereof, wherein:

R₁-R₉ are independently selected from hydrogen and deuterium, at least one of R₁-R₅ is deuterium, and with the proviso that when any of R₆, R₇, R₈, and R₉ are deuterium, at least one of R₁-R₅ are also deuterium.

In further embodiments, said pharmaceutically acceptable salt is selected from a hydrochloride, a hydrobromide, a sulfate, a formate, an acetate, a fumarate, a citrate, a tartrate, a mesylate, a tosylate, a besylate, and the like.

In specific embodiments, said pharmaceutically acceptable salt is a hydrochloride salt.

In certain embodiments, R₁ is deuterium.

In certain embodiments, R₂ is deuterium.

In certain embodiments, R₃ is deuterium.

In certain embodiments, R₄ is deuterium.

In certain embodiments, R₅ is deuterium.

In certain embodiments, R₆ is deuterium.

In certain embodiments, R₇ is deuterium.

In certain embodiments, R₈ is deuterium.

In certain embodiments, R₉ is deuterium.

In certain embodiments, R₄ and R₅ are deuterium.

In certain embodiments, R₄-R₅ and R₆-R₇ are deuterium.

In certain embodiments R₄-R₅ and R₈-R₉ are deuterium.

In certain embodiments R₄-R₅, R₆-R₇, and R₈-R₉ are deuterium.

Also provided herein are embodiments according to each of the embodiments above, wherein R₁-R₃ are deuterium.

Also provided herein are embodiments according to each of the embodiments above, wherein R₈-R₉ are hydrogen.

Also provided herein are embodiments according to each of the embodiments above, wherein all of R₆-R₉ are hydrogen.

Also provided herein are embodiments according to each of the embodiments above, wherein every other substituent among R₁-R₉ not specified as deuterium is hydrogen.

In certain embodiments are provided compounds as disclosed herein, wherein at least one of R₁-R₉ independently has deuterium enrichment of no less than about 1%. In certain embodiments are provided compounds as disclosed herein, wherein at least one of R₁-R₉ independently has deuterium enrichment of no less than about 10%. In certain embodiments are provided compounds as disclosed herein, wherein at least one of R₁-R₉ independently has deuterium enrichment of no less than about 50%, including about 55%, about 60%, about 65%, about 70%, about 75%, about 80%, about 85%, about 90%, about 95%, and about 100%. In certain embodiments are provided compounds as disclosed herein, wherein at least one of R₁-R₉ independently has deuterium enrichment of no less than about 90%, including about 92%, about 94%, about 96%, about 98%, and about 100%. In certain embodiments are provided compounds as disclosed herein, wherein at least one of R₁-R₉ independently has deuterium enrichment of no less than about 95%, including about 96%, about 97%, about 98%, about 99%, and about 100%. In certain embodiments are provided compounds as disclosed herein, wherein at least one of R₁-R₉ independently has deuterium enrichment of no less than about 98%, including about 99%, and about 100%.

Thus, as described herein, the compound of Formula I and/or the compound of Formula Ia does not encompass the compound of Formula II

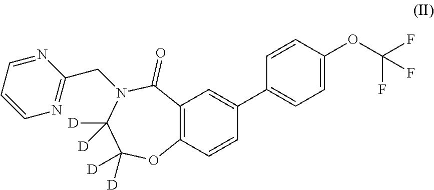 [SMILES: [2H]C1([2H])Oc2ccc(-c3ccc(OC(F)(F)F)cc3)cc2C(=O)N(Cc2ncccn2)C1([2H])[2H]]

or a pharmaceutically acceptable salt, ester, prodrug, or solvate thereof.

In other embodiments, provided is a compound or intermediate of Formula III or IV:

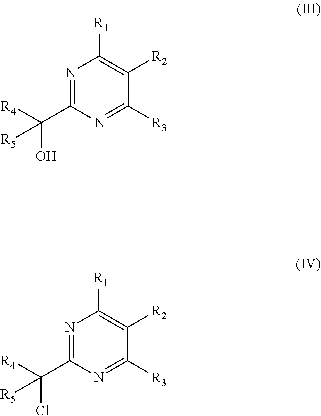

or a pharmaceutically acceptable salt, ester, prodrug, or solvate thereof, wherein:

R₁-R₅ are independently selected from hydrogen and deuterium; and at least one of R₁-R₅ is deuterium.

In certain embodiments, both R₄ and R₅ are deuterium.

In certain embodiments, at least one of R₁-R₃ is deuterium.

In certain embodiments, at least two of R₁-R₃ are deuterium.

In certain embodiments, all of R₁-R₃ are deuterium.

Certain compounds disclosed herein may possess useful late sodium current inhibiting activity, and may be used in the treatment or prophylaxis of a disorder in which late sodium current activity plays an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for inhibiting late sodium current activity. Other embodiments provide methods for treating a late sodium current-mediated disorder in a mammal and/or patient in need of such treatment, comprising administering to said patient a therapeutically effective amount of a compound or composition (of Formula I) according to the present invention. Also provided is the use of certain compounds disclosed herein for use in the manufacture of a medicament for the prevention or treatment of a disorder ameliorated by inhibiting late sodium current activity.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments are provided compounds as disclosed herein, wherein each position represented as D has deuterium enrichment of no less than about 1%. In certain embodiments are provided compounds as disclosed herein, wherein each position represented as D has deuterium enrichment of no less than about 10%. In certain embodiments are provided compounds as disclosed herein, wherein each position represented as D has deuterium enrichment of no less than about 50%. In certain embodiments are provided compounds as disclosed herein, wherein each position represented as D has deuterium enrichment of no less than about 90%. In certain embodiments are provided compounds as disclosed herein, wherein each position represented as D has deuterium enrichment of no less than about 95%. In certain embodiments are provided compounds as disclosed herein, wherein each position represented as D has deuterium enrichment of no less than about 98%.

In certain embodiments, said compound of Formula I is selected from the group consisting of:

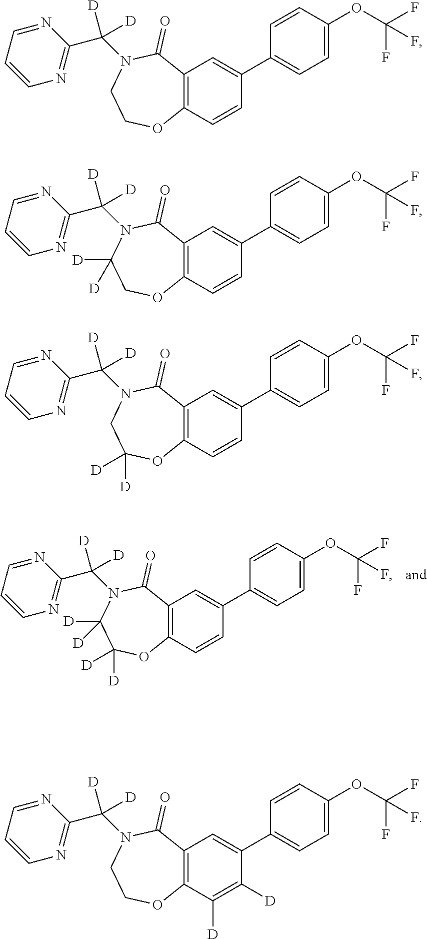 [SMILES: [2H]C([2H])(c1ncccn1)N1CCOc2ccc(-c3ccc(OC(F)(F)F)cc3)cc2C1=O ; [2H]C1([2H])COc2ccc(-c3ccc(OC(F)(F)F)cc3)cc2C(=O)N1C([2H])([2H])c1ncccn1 ; [2H]C1([2H])CN(C([2H])([2H])c2ncccn2)C(=O)c2cc(-c3ccc(OC(F)(F)F)cc3)ccc2O1 ; [2H]C([2H])(c1ncccn1)N1C(=O)c2cc(-c3ccc(OC(F)(F)F)cc3)ccc2OC([2H])([2H])C1([2H])[2H] ; [2H]c1c(-c2ccc(OC(F)(F)F)cc2)cc2c(c1[2H])OCCN(C([2H])([2H])c1ncccn1)C2=O]

It is noted that in any of the above enumerated compounds of Formula I, the aromatic rings (i.e. the pyrimidyl ring, the phenyl ring, and/or the fused phenyl ring) may also contain one or more deuterium.

In other embodiments, said compound of Formula I is selected from:

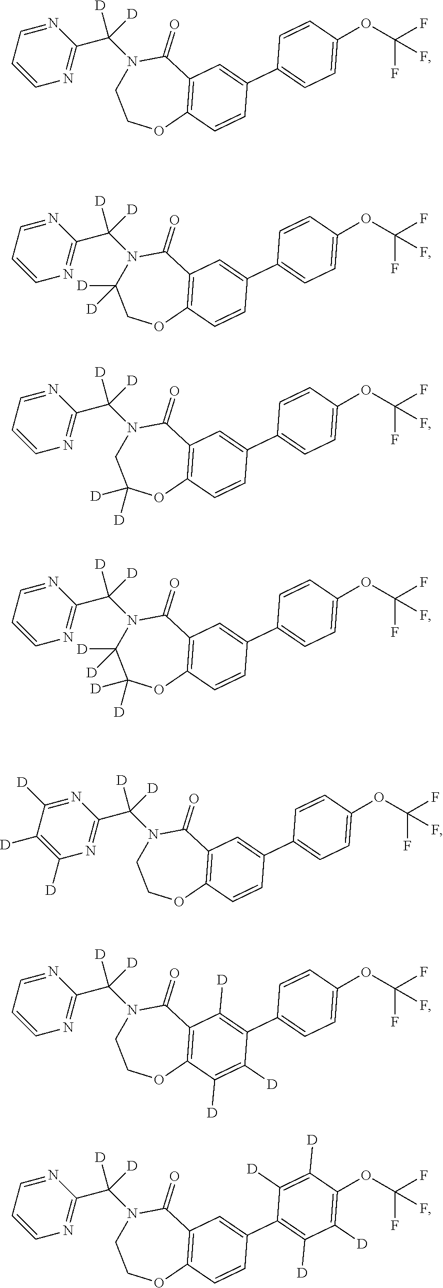

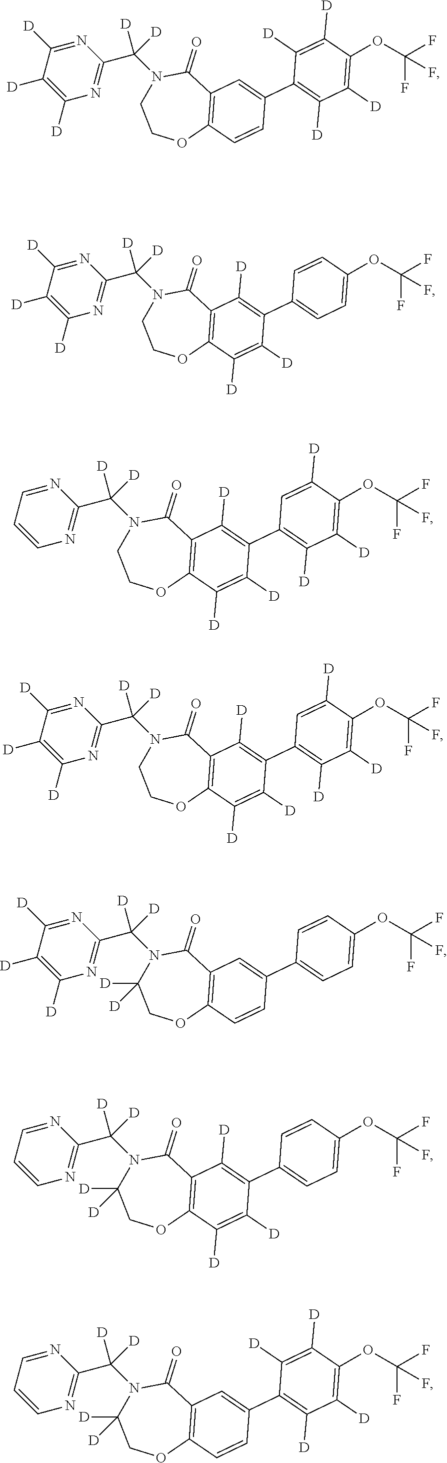

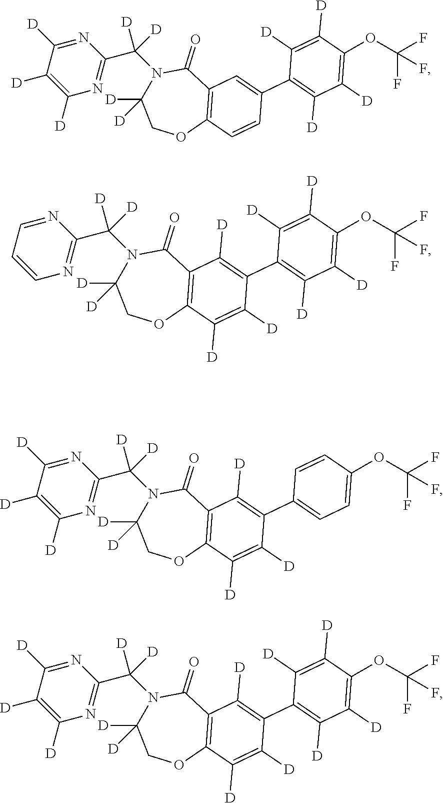 [SMILES: [2H]c1nc(C([2H])([2H])N2C(=O)c3cc(-c4c([2H])c([2H])c(OC(F)(F)F)c([2H])c4[2H])ccc3OCC2([2H])[2H])nc([2H])c1[2H] ; [2H]c1c([2H])c(-c2c([2H])c([2H])c3c(c2[2H])C(=O)N(C([2H])([2H])c2ncccn2)C([2H])([2H])CO3)c([2H])c([2H])c1OC(F)(F)F ; [2H]c1nc(C([2H])([2H])N2C(=O)c3c([2H])c(-c4ccc(OC(F)(F)F)cc4)c([2H])c([2H])c3OCC2([2H])[2H])nc([2H])c1[2H] ; [2H]c1nc(C([2H])([2H])N2C(=O)c3c([2H])c(-c4c([2H])c([2H])c(OC(F)(F)F)c([2H])c4[2H])c([2H])c([2H])c3OCC2([2H])[2H])nc([2H])c1[2H]]

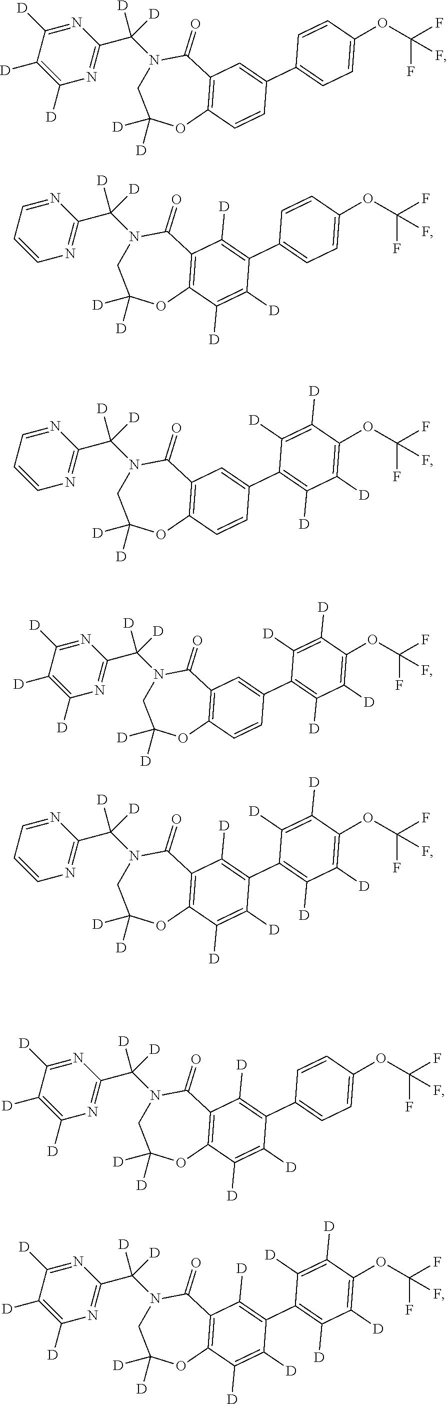

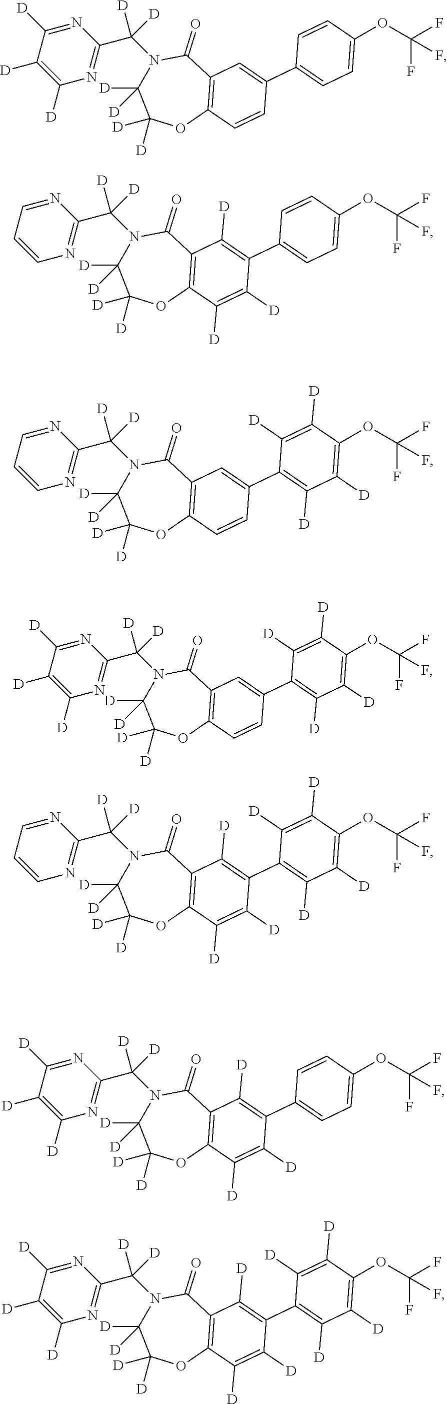

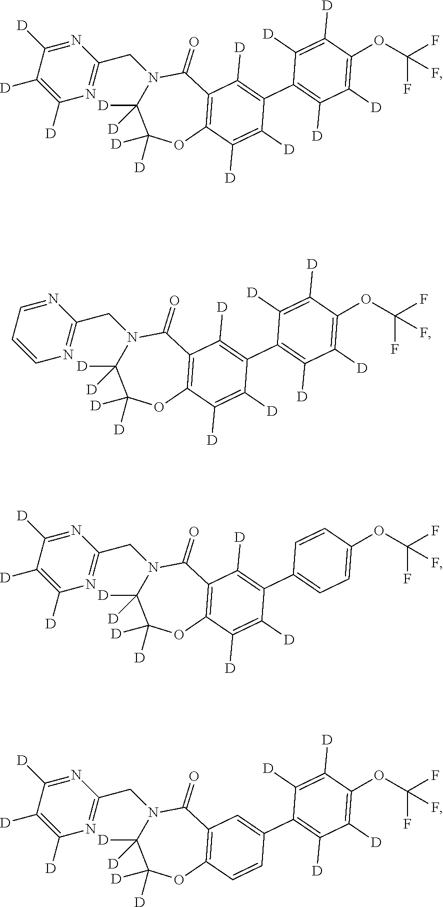 [SMILES: [2H]c1nc(CN2C(=O)c3c([2H])c(-c4c([2H])c([2H])c(OC(F)(F)F)c([2H])c4[2H])c([2H])c([2H])c3OC([2H])([2H])C2([2H])[2H])nc([2H])c1[2H] ; [2H]c1c([2H])c(-c2c([2H])c([2H])c3c(c2[2H])C(=O)N(Cc2ncccn2)C([2H])([2H])C([2H])([2H])O3)c([2H])c([2H])c1OC(F)(F)F ; [2H]c1nc(CN2C(=O)c3c([2H])c(-c4ccc(OC(F)(F)F)cc4)c([2H])c([2H])c3OC([2H])([2H])C2([2H])[2H])nc([2H])c1[2H] ; [2H]c1nc(CN2C(=O)c3cc(-c4c([2H])c([2H])c(OC(F)(F)F)c([2H])c4[2H])ccc3OC([2H])([2H])C2([2H])[2H])nc([2H])c1[2H]]

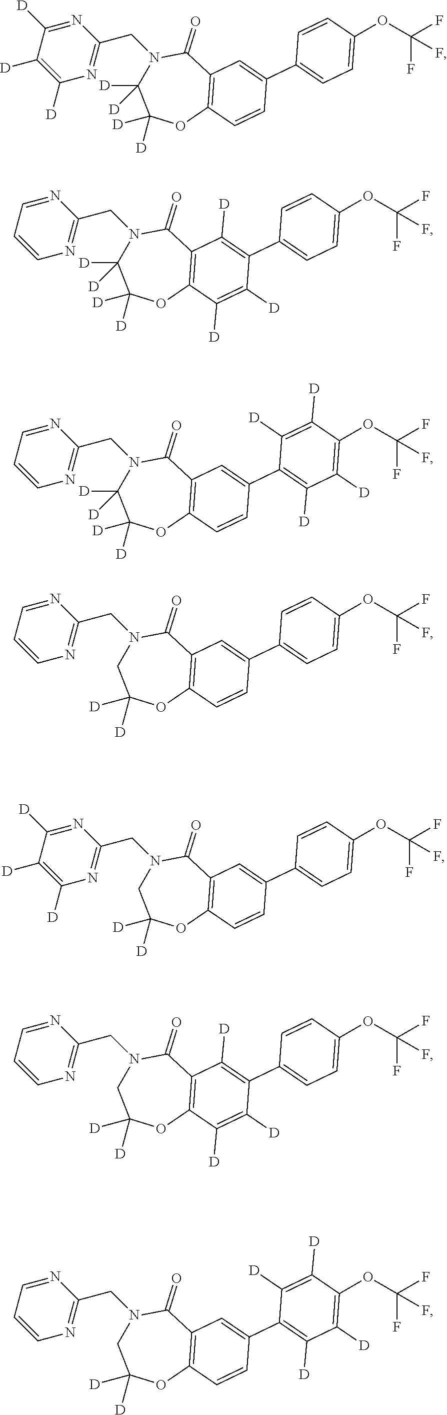

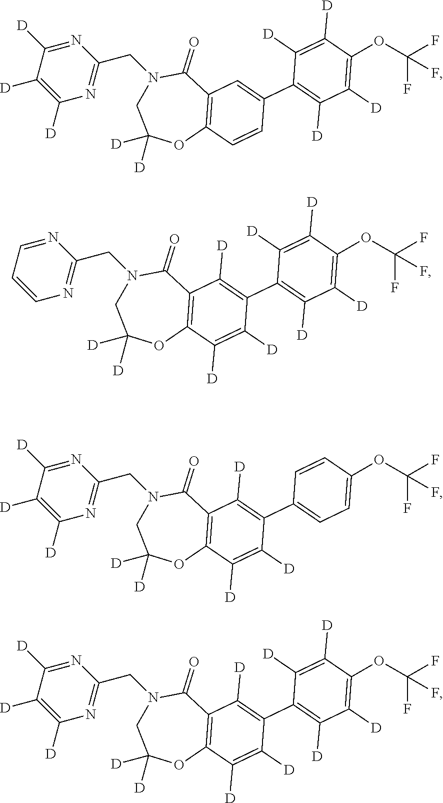 [SMILES: [2H]c1nc(CN2CC([2H])([2H])Oc3ccc(-c4c([2H])c([2H])c(OC(F)(F)F)c([2H])c4[2H])cc3C2=O)nc([2H])c1[2H] ; [2H]c1c([2H])c(-c2c([2H])c([2H])c3c(c2[2H])C(=O)N(Cc2ncccn2)CC([2H])([2H])O3)c([2H])c([2H])c1OC(F)(F)F ; [2H]c1nc(CN2CC([2H])([2H])Oc3c([2H])c([2H])c(-c4ccc(OC(F)(F)F)cc4)c([2H])c3C2=O)nc([2H])c1[2H] ; [2H]c1nc(CN2CC([2H])([2H])Oc3c([2H])c([2H])c(-c4c([2H])c([2H])c(OC(F)(F)F)c([2H])c4[2H])c([2H])c3C2=O)nc([2H])c1[2H]]

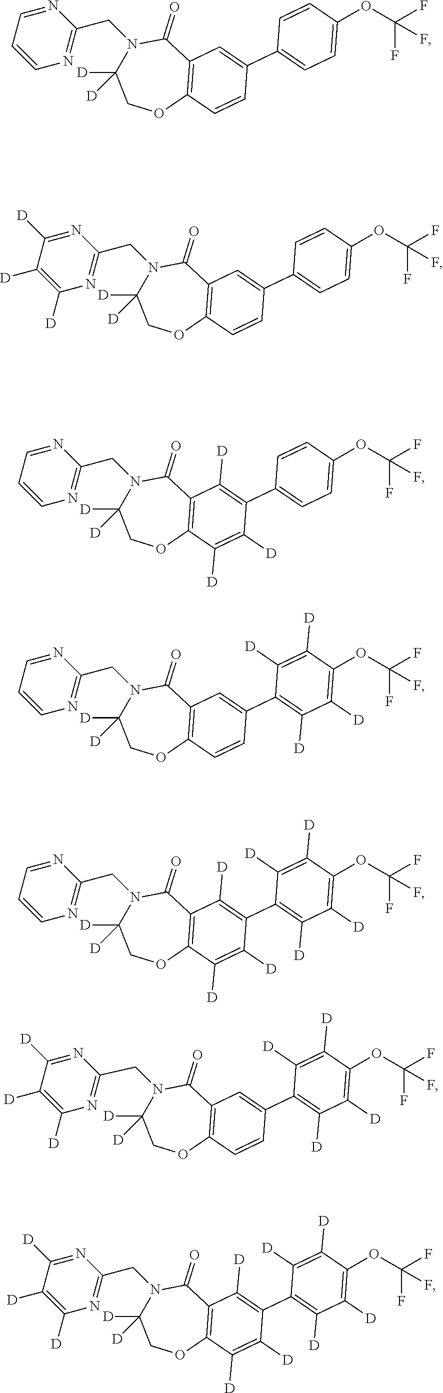

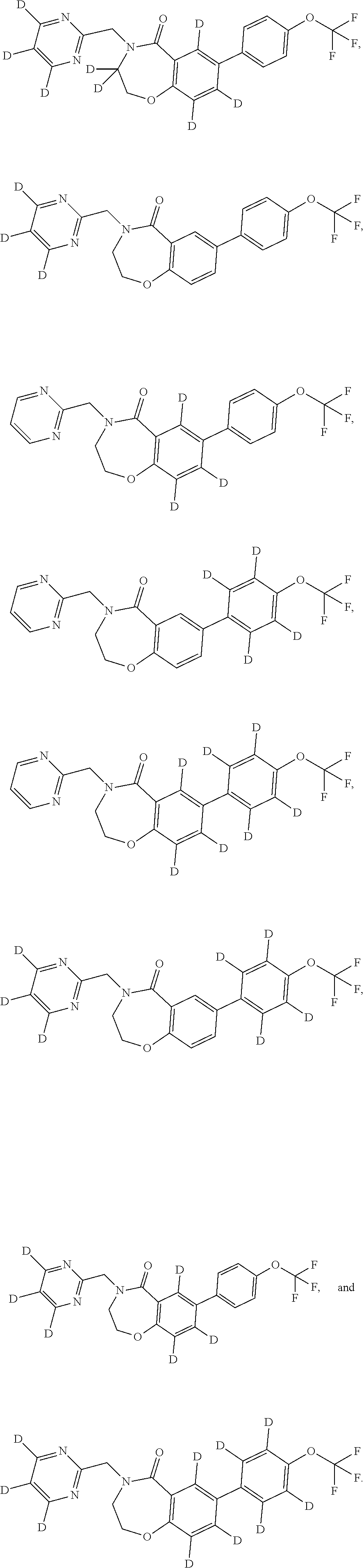

It is noted that in any of the above enumerated compounds of Formula I, the aromatic rings (i.e. the pyrimidyl ring, the phenyl ring, and/or the fused phenyl ring) may also contain one or more deuterium.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Also provided is a pharmaceutical composition comprising a compound as disclosed herein together with a pharmaceutically acceptable carrier.

Also provided is a pharmaceutical composition comprising a pharmaceutically acceptable carrier together with a compound of Formula I:

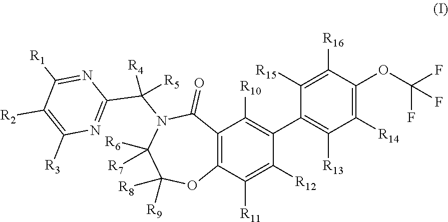

or a pharmaceutically acceptable salt, ester, prodrug, or solvate thereof, wherein:

R₁-R₁₆ are independently selected from the consisting of hydrogen and deuterium, and at least one of R₁-R₁₆ is deuterium, with the proviso that when all of R₆, R₇, R₈, and R₉ are deuterium, at least one of R₁-R₅ or R₁₀-R₁₆ are also deuterium, and the pharmaceutical composition has deuterium enrichment of at least 10% is at least one of the positions of R₁-R₁₆.

In certain embodiments, R₁ is deuterium.

In certain embodiments, R₂ is deuterium.

In certain embodiments, R₃ is deuterium.

In certain embodiments, R₄ is deuterium.

In certain embodiments, R₅ is deuterium.

In certain embodiments, R₆ is deuterium.

In certain embodiments, R₇ is deuterium.

In certain embodiments, R₈ is deuterium.

In certain embodiments, R₉ is deuterium.

In certain embodiments, R₁₀ is deuterium.

In certain embodiments, R₁₁ is deuterium.

In certain embodiments, R₁₂ is deuterium.

In certain embodiments, R₁₃ is deuterium.

In certain embodiments, R₁₄ is deuterium.

In certain embodiments, R₁₅ is deuterium.

In certain embodiments, R₁₆ is deuterium.

In specific embodiments, at least one of R₄-R₅ has deuterium enrichment of at least 10%.

In other embodiments, also provided is a pharmaceutical composition comprising a pharmaceutically acceptable carrier together with a compound of Formula Ia:

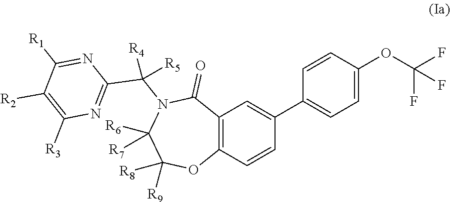

or a pharmaceutically acceptable salt, ester, prodrug, or solvate thereof, wherein:

R₁-R₉ are independently selected from hydrogen and deuterium, at least one of R₁-R₅ is deuterium, and with the proviso that when any of R₆, R₇, R₈, and R₉ are deuterium, at least one of R₁-R₅ are also deuterium.

In further embodiments, said pharmaceutically acceptable salt of Formula I and/or Formula Ia is selected from a mesylate, a tosylate, a besylate, a hydrochloride, a hydrobromide, a sulfate, a formate, an acetate, a fumarate, a citrate, a tartrate, and the like.

In specific embodiments, said pharmaceutically acceptable salt is a hydrochloride salt.

In certain embodiments, R₁ is deuterium.

In certain embodiments, R₂ is deuterium.

In certain embodiments, R₃ is deuterium.

In certain embodiments, R₄ is deuterium.

In certain embodiments, R₅ is deuterium.

In certain embodiments, R₆ is deuterium.

In certain embodiments, R₇ is deuterium.

In certain embodiments, R₈ is deuterium.

In certain embodiments, R₉ is deuterium.

In specific embodiments, at least one of R₄-R₅ has deuterium enrichment of at least 10%.

In some embodiments, the compounds of Formula I and/or Formula Ia are effective in the treatment of conditions or diseases known to respond to administration of late sodium channel blockers, including but not limited to cardiovascular diseases such as atrial and ventricular arrhythmias, including atrial fibrillation, Prinzmetal's (variant) angina, stable angina, unstable angina, ischemia and reperfusion injury in cardiac kidney, liver, and the brain, exercise induced angina, pulmonary hypertension, congestive heart disease including diastolic and systolic heart failure, and myocardial infarction. In other embodiments, the compounds of Formula which function as late sodium channel blockers in the treatments of diseases including cardiomyopathy, hypertrophic cardiomyopathy, angina, heart failure, atrial fibrillation (AF), ischaemic heart disorders, myocardial ischemia, arrhythmias, congestive heart failure, myocardial infarction, long QT syndrome, diabetes, inflammatory diseases, and proliferative diseases. In still further embodiments, the compounds of Formula I and/or Formula Ia which function as late sodium channel blockers may be used in the treatment of diseases affecting the neuro-muscular system which result in pain, itching, seizures, or paralysis, or in the treatment of diabetes or reduced insulin sensitivity, and disease states related to diabetes, such as diabetic peripheral neuropathy.

Certain compounds of Formula I and/or Formula Ia may also possess a sufficient activity in modulating neuronal sodium channels, and may have pharmacokinetic properties such that they may be active with regard to the central and/or peripheral nervous system. Consequently, some compounds of Formula I and/or Formula Ia may also be of use in the treatment of epilepsy or pain or itching or headache of a neuropathic origin.

Also provided is a method of treating or preventing a late sodium current-mediated disorder, the method comprising administering, to a mammal in need thereof, a therapeutically effective amount of a compound of Formula I and/or Formula Ia or administering a pharmaceutical composition comprising a compound of Formula I and/or Formula Ia. In other embodiments, provided is a method of treating a disease selected from acute coronary syndrome, peripheral arterial disease, intermittent claudication, Prinzmetal's (variant) angina, stable angina, unstable angina, ischemia, recurrent ischemia, reperfusion injury, exercise induced angina, pulmonary hypertension, congestive heart disease including diastolic and systolic heart failure, myocardial infarction, cardiomyopathy, hypertrophic cardiomyopathy, heart failure, atrial fibrillation (AF), ischaemic heart disorders, myocardial ischemia, arrhythmias, congestive heart failure, long QT syndrome, diabetes, reduced insulin sensitivity, diseases affecting the neuro-muscular system which result in pain, itching, seizures, or paralysis, inflammatory diseases, diabetic peripheral neuropathy, and proliferative diseases, the method comprising administering a therapeutically effective amount of at least one compound of Formula I and/or Formula Ia to a mammal in need thereof.

In certain embodiments, the method further results in at least one effect selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the administration in said subject per dosage unit thereof as compared to the non-isotopically enriched compound.

In certain embodiments, the method further results in at least two effects selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the administration in said subject per dosage unit thereof as compared to the non-isotopically enriched compound.

In certain embodiments, the method effects a decreased metabolism of the compound per dosage unit thereof by at least one polymorphically-expressed cytochrome P₄₅₀ isoform in the subject, as compared to the corresponding non-isotopically enriched compound.

In certain embodiments, the cytochrome P₄₅₀ isoform is selected from the group consisting of CYP2C8, CYP2C9, CYP2C19, CYP2D6, and CYP3A4.

In certain embodiments, said compound is characterized by decreased inhibition of at least one cytochrome P₄₅₀ or monoamine oxidase isoform in said subject per dosage unit thereof as compared to the non-isotopically enriched compound.

In certain embodiments, said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

In certain embodiments, the method reduces a deleterious change in a diagnostic hepatobiliary function endpoint, as compared to the corresponding non-isotopically enriched compound.

In certain embodiments, the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

With respect to the terms used in this disclosure, the following definitions are provided.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

As used herein, the term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

As used herein, the term “is/are deuterium,” when used to describe a given position in a molecule such as R₁-R₁₆ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

As used herein, the term “isotopic enrichment” refers to the percentage of incorporation of a less prevalent isotope of an element at a given position in a molecule in the place of the more prevalent isotope of the element.

As used herein, the term “non-isotopically enriched” refers to a molecule in which the percentages of the various isotopes are substantially the same as the naturally occurring percentages.

Asymmetric centers may exist in the compounds disclosed herein. Compounds of the present invention containing an asymmetrically substituted atom may be isolated in optically active or racemic forms. It is well known in the art how to prepare optically active forms, such as by resolution of racemic forms or by synthesis from optically active materials. Asymmetric centers are designated by the symbols “R” or “S,” depending on the configuration of substituents around the chiral carbon atom. It should be understood that the invention encompasses all chiral, diastereomeric, racemic forms, and all geometric isomeric forms, and mixtures thereof, unless the specific stereochemistry or isomeric form is specifically indicated. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

As used herein, the term “substituted” means that any one or more hydrogens on the designated atom or ring is replaced with a selection from the indicated group, e.g. deuterium, provided that the designated atom's normal valency is not exceeded.

As used herein, the term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

As used herein, the term “disorder” is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

As used herein, the terms “treat,” “treating,” and “treatment” are meant to include alleviating or abrogating a disorder or one or more of the symptoms associated with a disorder; or alleviating or eradicating the cause(s) of the disorder itself. In some embodiments, treating refers to inhibiting the disease-state, i.e., arresting its development and/or relieving the disease-state, i.e., causing regression of the disease state.

As used herein, the terms “prevent,” “preventing,” and “prevention” refer to a method of delaying or precluding the onset of a disorder; and/or its attendant symptoms, barring a subject from acquiring a disorder or reducing a subject's risk of acquiring a disorder. In some embodiments, preventing refers to precluding a disease-state from occurring in a mammal, in particular, when such mammal is pre-disposed to the disease-state but has not yet been diagnosed as having it.

As used herein, the term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

As used herein, the term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human and/or a canine and/or a feline patient.

As used herein, the term “combination therapy” refers to the administration of two or more therapeutic agents to treat (or prevent) a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment (or prevention) regimen will provide beneficial effects of the drug combination in treating the disorders described herein.

As used herein, the term “late sodium current-mediated disorder,” refers to a disorder that is characterized by abnormal late sodium current activity, or normal late sodium current activity that when modulated ameliorates other abnormal biochemical processes. A late sodium current-mediated disorder may be completely or partially mediated by modulating late sodium current activity. In particular, a late sodium current-mediated disorder is one in which inhibition of late sodium current activity results in some effect on the underlying disorder, e.g., administration of a late sodium current disorder inhibitor results in some improvement in at least some of the patients being treated.

As used herein, the terms “late sodium current inhibitor” or “late sodium channel inhibitor” or “late sodium current blocker” or “late sodium channel blocker” refers to the ability of a compound disclosed herein to alter the function of the late sodium current. Blockade (or inhibition) of the late sodium current, I_{Na}, reduces the sodium and calcium overload that follows ischemia. This improves myocardial relaxation and reduces left ventricular diastolic stiffness, in turn enhancing myocardial contractility and perfusion. As used herein, the terms “inhibiting late sodium current activity” or “inhibition of late sodium current activity” or “blocking late sodium current activity” or the like refer to altering the function of the late sodium current by administering a late sodium current inhibitor.

As used herein, the terms “therapeutically acceptable” and “pharmaceutically acceptable” are used interchangeably and refer to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

As used herein, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio.

As used herein, the terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

As used herein, the terms “drug,” “therapeutic agent,” and “chemotherapeutic agent” refer to a compound, or a pharmaceutical composition thereof, which is administered to a subject for treating, preventing, or ameliorating one or more symptoms of a disorder.

As used herein, the term “release controlling excipient” refers to an excipient whose primary function is to modify the duration or place of release of the active substance from a dosage form as compared with a conventional immediate release dosage form.

As used herein, the term “nonrelease controlling excipient” refers to an excipient whose primary function do not include modifying the duration or place of release of the active substance from a dosage form as compared with a conventional immediate release dosage form.

As used herein, the term “prodrug” refers to a compound functional derivative of the compound as disclosed herein and is readily convertible into the parent compound in vivo. The term “prodrug” denotes a compound which, upon administration to a subject, undergoes chemical conversion by metabolic or chemical processes to yield a compound of the formula, and/or a salt and/or solvate thereof. For example, compounds containing a carboxy group can form physiologically hydrolyzable esters which serve as prodrugs by being hydrolyzed in the body to yield formula compounds per se. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The term “prodrug” as employed herein includes esters and carbonates formed by reacting compounds of formula I with alkyl, alkoxy, or aryl substituted acylating agents employing procedures known to those skilled in the art to generate acetates, pivalates, methyl carbonates, benzoates, and the like.

The compounds disclosed herein can exist as therapeutically acceptable salts or pharmaceutically acceptable salts. As used herein, the terms “therapeutically acceptable salt” and “pharmaceutically acceptable salt” are used interchangeably and represent salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts.

The term pharmaceutically acceptable salt includes acid addition salts. There are formed, for example, with strong inorganic acids, such as mineral acids or hydrohalic acids, with strong organic carboxylic acid, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, benzoic acid, or organic sulfonic acids.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecyl sulfuric acid, ethane-1,2-disulfonic acid, ethanesulfonic acid, 2-hydroxy-ethanesulfonic acid, formic acid, fumaric acid, phthalic acid, terephthalic acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Such pharmaceutically acceptable salts may refer to basic salts formed with inorganic and organic bases. Such salts include ammonium salts; alkali metal salts, such as lithium, sodium, and potassium salts; alkaline earth metal salts, such as calcium and magnesium salts; salts with organic bases, such as amine like salts (e.g., dicyclohexylamine salt, benzathine, N-methyl-D-glucamine, and hydrabamine salts); and salts with amino acids like arginine, lysine, and the like; and zwitterions, the so-called “inner salts.” Nontoxic, pharmaceutically acceptable salts are preferred, although other salts are also useful, e.g., in isolating or purifying the product.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In specific embodiments, hydrochloric acid is used for the preparation of a pharmaceutically acceptable salt. The resulting salt is, thus, a hydrochloride. In further embodiments, the pharmaceutically acceptable salt is selected from a mesylate, a tosylate, a besylate, a hydrobromide, a sulfate, a formate, an acetate, a fumarate, a citrate, a tartarate, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

As used herein, the terms “therapeutically effective amount” and “pharmaceutically effective amount” are intended to include an amount of a compound of the present invention alone or an amount of the combination of compounds claimed or an amount of a compound of the present invention in combination with other active ingredients effective to treat or prevent a late sodium current-mediated disorder.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. Such carriers enable the pharmaceutical compositions to be formulated as tablets, pills dragees, capsules, liquids, gels, syrups, slurries, suspensions, and the like, for ingestion, application, or inhalation by the patient. In addition to the active ingredients (e.g. the compounds of structural Formula I), the pharmaceutical compositions may contain suitable pharmaceutically acceptable carriers comprising excipients and auxiliaries which facilitate processing of the active compounds into preparations which can be used pharmaceutically. Techniques for formulation and administration are known in the art.

The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art.

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. Such compositions may be present in the form of a gel, paste, ointment, cream, lotion, liquid suspension, dispersion, emulsions, micro-emulsions, microcapsules, microparticles, vesicular dispersions, and the like.

The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment (i.e., administration) on a long-term basis upon any recurrence of symptoms.

Disclosed herein are methods of treating a late sodium current-mediated disorder comprising administering to a subject having or suspected of having such a disorder, a therapeutically effective amount of a compound as disclosed herein or a pharmaceutically acceptable salt, solvate, or prodrug thereof.

Without intending to be bound by theory, it is thought that the compounds of Formula I target and block the late sodium current. Thus, one or more embodiments are directed to the inhibition or blocking of the late sodium current in order to treat or prevent a late sodium current-mediated disorder.

Late sodium current-mediated disorders, include, and are not limited to, acute coronary syndrome, peripheral arterial disease, intermittent claudication, Prinzmetal's (variant) angina, stable angina, unstable angina, ischemia, recurrent ischemia, reperfusion injury, exercise induced angina, pulmonary hypertension, congestive heart disease including diastolic and systolic heart failure, myocardial infarction, cardiomyopathy, hypertrophic cardiomyopathy, heart failure, atrial fibrillation (AF), atrial premature beats (APBs), ischaemic heart disorders, myocardial ischemia, arrhythmias, congestive heart failure, long QT syndrome, diabetes, reduced insulin sensitivity, diseases affecting the neuro-muscular system which result in pain, itching, seizures, or paralysis, inflammatory diseases, diabetic peripheral neuropathy, and proliferative diseases, and/or any disorder which can lessened, alleviated, or prevented by administering a late sodium current inhibitor/blocker.

In certain embodiments, a method of treating a late sodium current-mediated disorder comprises administering to the subject a therapeutically effective amount of a compound as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described the art.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol. Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, CYP2D6, CYP3A4, and CYP3A5.

The metabolic activities of liver microsomes, cytochrome P₄₅₀ isoforms, and monoamine oxidase isoforms are measured by the methods described herein.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

Combination Therapy

Patients being treated by administration of the late sodium channel blockers of Formula I often exhibit diseases or conditions that may benefit from treatment with other therapeutic agents. These diseases or conditions can be of cardiovascular nature or can be related to pulmonary disorders, metabolic disorders, gastrointestinal disorders, and the like. Additionally, some cardiovascular patients being treated by administration of the late sodium channel blockers of Formula I and/or Formula Ia exhibit conditions that can benefit from treatment with therapeutic agents that are antibiotics, atlalgesics, and/or antidepressants and anti-anxiety agents.

The present invention includes within its scope pharmaceutical compositions comprising, as an active ingredient, a therapeutically effective amount of at least one compound of Formula I and/or Formula Ia, alone or in combination with a pharmaceutical carrier or diluent. Optionally, compounds of the present invention can be used alone, in combination with other compounds of the invention (i.e. additional compounds of Formula I and/or Formula Ia), or in combination with one or more other therapeutic agent(s).

In certain embodiments, the method additionally comprises administering, or co-administering, an additional therapeutic agent in combination with one or more compounds of Formula I and/or Formula Ia. The compounds disclosed herein may also be combined or used in combination with other agents useful in the treatment or prevention of late sodium current-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

Thus, in another aspect, embodiments provide methods for treating late sodium current-mediated disorders in a human or animal comprising administering to said subject an amount of a compound of Formula I and/or Formula Ia disclosed herein effective to reduce or prevent said late sodium current-mediated disorder in the subject, in combination with at least one additional therapeutic agent for the treatment of said disorder. In a related aspect, embodiments provide pharmaceutical compositions comprising at least one compound disclosed herein in combination with one or more additional therapeutic agents for the treatment of late sodium current-mediated disorders.

The choice of additional therapeutic agent may be made from any additional therapeutic agent known to be useful for co-administration with a late sodium current inhibitor (i.e. late sodium channel blocker), such as eleclazine or ranolazine. Examples of conditions and disease that may be treated with a compound of Formula I in combination with an additional therapeutic agent are (1) a cardiovascular disease or condition requiring administration of an agent selected from a calcium channel blocker, a beta-blocker, a nitrate, a remodeling agent (e.g. metoprolol tartrate, enalapril maleate, etc.), pyridoxal-5′-phosphate, a sterol absorption inhibitor, a sodium-hydrogen exchanger type-1 inhibitor, an aldosterone antagonist (e.g. eplerenone), an HMG CoA reductase-inhibitor, and an adenosine A-3 receptor agonist; (2) diabetes using an HMG CoA reductase inhibitor, a sterol absorption inhibitor, or a cholesterol ester transfer protein (CETP) inhibitor; (3) obesity using an HMG CoA reductase inhibitor, a sterol absorption inhibitor, or a cholesterol ester transfer protein (CETP) inhibitor; (4) high serum cholesterol using an HMG CoA reductase inhibitor, or a cholesterol ester transfer protein (CETP) inhibitor; (5) viral infections using a quinolone or a derivative or an intermediate thereof; (6) endothelial dysfunction using an HMG CoA reductase inhibitor; (7) inflammatory diseases, proliferative diseases, or wound treatment using a UCP inhibitor, or a Fas inhibitor; and (8) proliferative diseases using a chemotherapeutic agent.

Cardiovascular related diseases or conditions that can benefit from a combination treatment of the late sodium current inhibitors (late sodium channel blockers) of Formula I and/or Formula Ia with other therapeutic agents include, without limitation, angina including stable angina, unstable angina (UA), exercised-induced angina, variant angina, arrhythmias, intermittent claudication, myocardial infarction including non-STE myocardial infarction (NSTEMI), pulmonary hypertension including pulmonary arterial hypertension and chronic thromboembolic pulmonary hypertension, heart failure including congestive (or chronic) heart failure and diastolic heart failure and heart failure with preserved ejection fraction (diastolic dysfunction), acute heart failure, or recurrent ischemia.

Therapeutic agents suitable for treating cardiovascular related diseases or conditions include anti-anginals, heart failure agents, antithrombotic agents, antiarrhythmic agents, antihypertensive agents, and lipid lowering agents.

In some embodiments, the late sodium current inhibitors of Formula I and/or Formula Ia are co-administered with one or more of eleclazine or ranolazine.

Anti-anginals include beta-blockers, calcium channel blockers, and nitrates. Examples of beta-blockers include, but are not limited to, metoprolol (Lopressor®, Toprol® XL), carteolol (Cartrol®), acebutolol (Sectral®), betaxolol (Kerlone®), atenolol (Tenormin®), bisoprolol/hydrochlorothiazide (Ziac®), bisoprolol (Zebeta®), esmolol (Brevibloc®), labetalol (Normodyne®, Trandate®), nadolol (Corgard®), propranolol (Inderal®), sotalol (Betapace®), and timolol (Blocadren®).

Examples of nitrates include, but are not limited to, nitroglycerin, nitrate patches, isosorbide dinitrate, and isosorbide-5-mononitrate.

Examples of calcium channel blockers include, but are not limited to, bepridil (Vascor®), amlodipine (Norvasc®, Lotrel®), diltiazem (Cardizem®, Tiazac®), nifedipine (Adalat®, Procardia®), felodipine (Plendil®), nisoldipine (Sular®), nimodipine (Nimotop®), verapamil (Verelan®, Calan®, Isoptin®), and nicardipine.

Agents used to treat heart failure include ACE inhibitors, diurectics, vasodilators, and cardiac glycosides. Examples of ACE inhibitors include, but are not limited to, benazepril (Lotensin®), captopril (Capoten®), enalapril (Vasotec®), fosinopril (Monopril®), Lisinopril Zestril®), moexipril (Univasc®), perindopril (Aceon®), quinapril (Accupril®), ramipril (Altace®), and trandolapril (Mavik®).

Examples of diuretics include, but are not limited to, furosemide (Lasix®), metolazone (Zaroxolyn®), bumetanide (Bumex®), spironolactone (Aldactone®), eplerenone (Inspra®), and hydrochlorothiazide.

Examples of vasodilators include, but are not limited to, hydralazine, diazoxide, prazosin, clonidine, and methyldopa. Nitrates, ACE inhibitors, potassium channel activators, and calcium channel blockers may also act as vasodilators.

Examples of cardiac glycosides include, but are not limited to, digoxin, digitoxin, and digitalis.

Examples of antithrombotics include, but are not limited to, platelet inhibitors, anticoagulants, and thrombolytic agents.

Examples of platelet inhibitors include, but are not limited to, clopidogrel (Plavix®), ticlopidine, acetyl salicylic acid (aspirin), prasugrel (Effient®), cilostazol, dipyridamole, persantine sulfinpyrazone, indomethacin, and glycoprotein 11b/11a inhibitors (such as abciximab, tirofiban, and eptifibatide (Integrelin®).

Examples of anticoagulants include, but are not limited to, warfarin (Coumadin®), unfractionated heparin, low molecular weight heparin, danaparoid, lepirudin, argatroban, bivalirudin, apixaban (Eliquis®), rivaroxaban, and edoxaban.

Examples of thrombolytic agents include, but are not limited to, tissue plasminogen activator (t-PA), tenecteplase (TNK), streptokinase, and urokinase.

Antiarrhymthmic agents include, but are not limited to, quinidine, procainamide, amiodarone, dronedarone, lidocaine, and propafenone.

Antihypertensive agents include, but are not limited to, alpha-1-adrenergic antagonists, such as prazosin (Minipress®), doxazosin mesylate (Cardura®), prazosin hydrochloride (Minipress®), prazosin, polythiazide (Minizide®), and terazosin hydrochloride (Hytrin®); beta-adrenergic antagonists, such as propranolol (Inderal®), nadolol (Corgard®), timolol (Blocadren®), metoprolol (Lopres-Sor®), and pindolol (Visken®); central alpha-adrenoceptor agonists, such as clonidine hydrochloride (Catapres®), clonidine hydrochloride and chlorthalidone (Clorpres®, Combipres®), guanabenz Acetate (Wytensin®), guanfacine hydrochloride (Tenex®), methyldopa (Aldomet®), methyldopa and chlorothiazide (Aldoclor®), methyldopa and hydrochlorothiazide (Aldoril®); combined alpha/beta-adrenergic antagonists, such as labetalol (Normodyne®, Trandate®), carvedilol (Coreg®); adrenergic neuron blocking agents, such as guanethidine (Ismelin®), reserpine (Serpasil®); central nervous system-acting antihypertensives, such as clonidine (Catapres®), methyldopa (Aldomet®), guanabenz (Wytensin®); anti-angiotensin II agents; ACE inhibitors, such as perindopril (Aceon®) captopril (Capoten®), enalapril (Vasotec®), lisinopril (Prinivil®, Zestril®); angiotensin-II receptor antagonists, such as candesartan (Atacand®), eprosartan (Teveten®), irbesartan (Avapro®), losartan (Cozaar®), telmisartan (Micardis®), valsartan (Diovan®); calcium channel blockers, such as verapamil (Calan®, Isoptin®), diltiazem (Cardizem®), nifedipine (Adalat®, Procardia®); diuretics; direct vasodilators, such as nitroprusside (Nipride®), diazoxide (Hyperstat® IV), hydralazine (Apresoline®), minoxidil (Loniten®), verapamil; and potassium channel activators, such as aprikalim, bimakalim, cromakalim, emakalim, nicorandil, and pinacidil.

Lipid lowering agents can include, but are not limited to bezafibrate (Bezalip®), ciprofibrate (Modalim®), and statins, such as atorvastatin (Lipitor®), fluvastatin (Lescol®), lovastatin (Mevacor®, Altocor®), mevastatin, pitavastatin (Livalo®, Pitava®), pravastatin (Lipostat®), rosuvastatin (Crestor®), and simvastatin (Zocor®).

In some embodiments, a patient presenting with an acute cardiovascular disease event may suffer from secondary medical conditions such as one or more of a metabolic disorder, a pulmonary disorder, a peripheral vascular disorder, or a gastrointestinal disorder. Such patients may benefit from a treatment of a combination therapy comprising administration to the patient one or more compounds of Formula I and/or Formula Ia in combination with at least one additional therapeutic agent.

As used herein, examples of metabolic disorders include, but are not limited to, diabetes (including type I and type II diabetes), metabolic syndrome, dyslipidemia, obesity, glucose intolerance, polycystic ovarian syndrome (PCOS), hypertension, elevated serum cholesterol, and elevated triglycerides.

Examples of therapeutic agents that may be used to treat metabolic disorders included, but are not limited to, antihypertensive agents, lipid lowering agents, insulin, sulfonylureas, biguanides, alpha-glucosidase inhibitors, and incretin mimetics.

As used herein, the term “pulmonary disorder” refers to any disease or condition related to the lungs. Examples of pulmonary disorders include, but are not limited to, asthma, chronic obstructive pulmonary disease (COPD), pulmonary hypertension, emphysema, and bronchitis.

Examples of therapeutic agents that may be used to treat pulmonary disorders include, but are not limited to, corticosteroids, bronchodilators, and electrolyte supplements.

As used herein, the term “gastrointestinal disorder” refers to diseases and conditions associated with the gastrointestinal tract, including, but not limited to, gastroesophageal reflux disease (GERD), inflammatory bowel disease (IBD), gastroenteritis, peptic ulcer disease, pancreatitis, and gastritis.

Examples of therapeutic agents that may be used to treat gastrointestinal disorders include, but are not limited to, proton pump inhibitors, H₂ blockers, prostaglandins, and antacids.

As used herein, the term “peripheral vascular disorder” refers to disorders related to the blood vessels (arteries and veins) located outside the heart and the brain, including, but not limited to, peripheral arterial disease (PAD).

In other embodiments, a patient presenting with an acute cardiovascular disease event may suffer from secondary medical conditions and/or symptoms that may benefit from the administration of one or more additional therapeutic agents including, but not limited to antibiotics, analgesics, antidepressants, and anti-anxiety agents.

The methods of combination therapy include co-administration of a formulation containing one or more late sodium current inhibitor of Formula I and/or Formula Ia and at least one additional therapeutic agent, essentially contemporaneous administration of more than one formulation comprising the late sodium current inhibitor of Formula I and the additional therapeutic agents or agents, and consecutive administration

General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds disclosed herein can be prepared by methods known to one of skill in the art and routine modifications thereof, and/or following procedures similar to those described in the Example section herein and routine modifications thereof, and/or procedures found in U.S. Pat. No. 8,586,732, U.S. Pat. No. 8,697,863, U.S. Pat. No. 8,962,610, U.S. Pat. No. 9,193,694, U.S. 2015/239904, and U.S. 2016/096846.

Compounds of Formula I (and, similarly, the compounds of Formula Ia) may be prepared as shown in the following reaction schemes and the description thereof, as well as relevant literature procedures that may be used by one skilled in the art. Exemplary reagents and procedures for these reactions appear hereinafter and in the working Examples. Any position shown as hydrogen may optionally be replaced with deuterium.

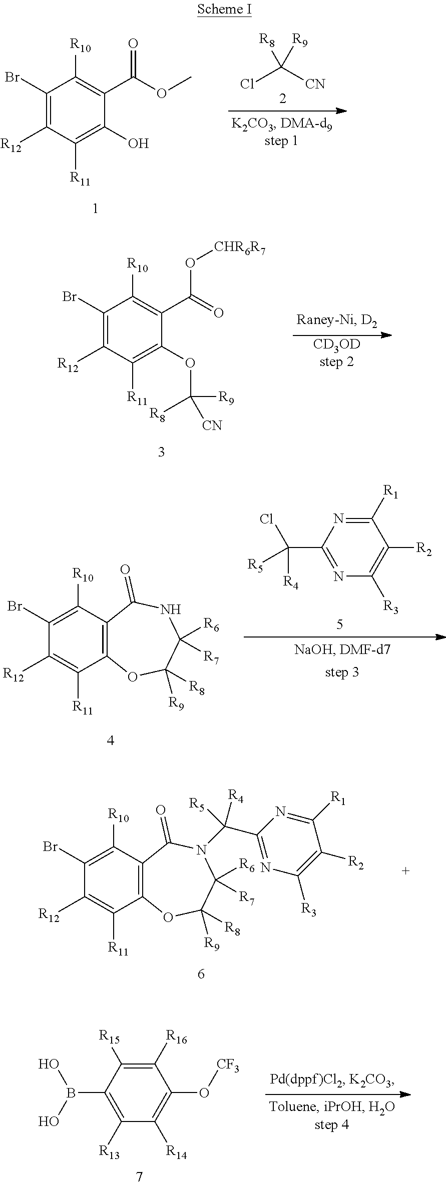

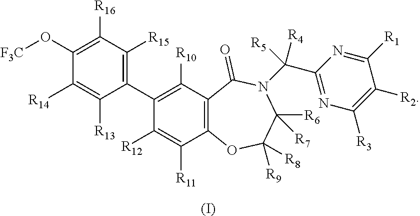

Deuterium can be incorporated into different positions synthetically, according to the synthetic procedures as shown in Scheme I, by using appropriate deuterated intermediates. For example, to introduce deuterium at one or more positions of R₁₀-R₁₂, compound 1 with the corresponding deuterium substitutions can be used. To introduce deuterium at R₁-R₅, compound 5 with the corresponding deuterium substitutions can be used. To introduce deuterium at one or more positions of R₁₃-R₁₆, compound 7 with the corresponding deuterium substitutions can be used. To introduce deuterium at R₆-R₉, compound 2 with the corresponding deuterium substitutions and deuterated DMA can be used.

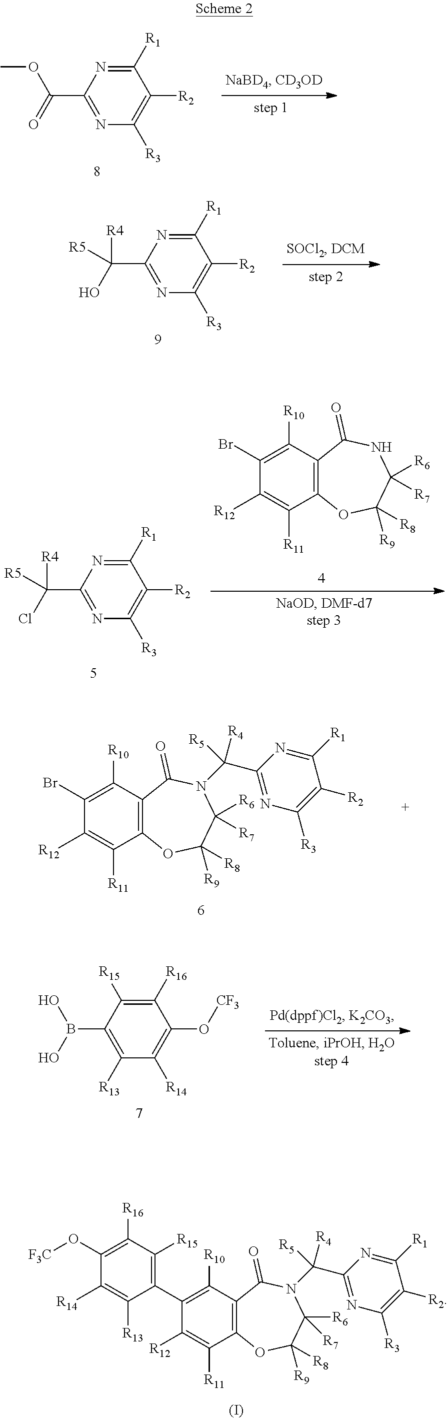

Deuterium can be incorporated into different positions synthetically, according to the synthetic procedures as shown in Scheme II, by using appropriate deuterated intermediates. For example, to introduce deuterium at one or more positions of R₁-R₃, compound 8 with the corresponding deuterium substitutions can be used. To introduce deuterium at R₄-R₅, deuterated sodium borohydride and deuterated methanol can be used. To introduce deuterium at one or more positions of R₆-R₁₂, compound 4 with the corresponding deuterium substitutions can be used. To introduce deuterium at R₁₃-R₁₆, compound 7 with the corresponding deuterium substitutions can be used.

Deuterium can be incorporated into various positions via proton-deuterium exchange reactions catalyzed by transition metals such palladium, rhodium, platinum. For example, to introduce deuterium at R₁-R₃ and/or at R₁₀-R₁₆, these protons may be replaced with deuterium selectively or non-selectively through a proton-deuterium exchange method known in the art.

One or more embodiments provide a compound or intermediate of Formula III and/or a compound of intermediate of Formula IV:

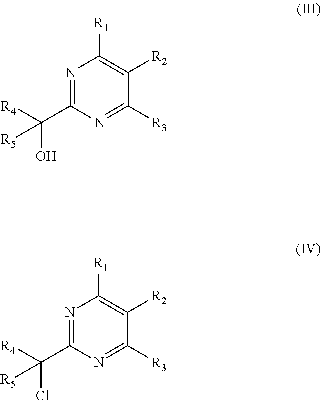

or a pharmaceutically acceptable salt, ester, prodrug, or solvate thereof, wherein:

R₁-R₅ are independently selected from hydrogen and deuterium; and at least one of R₁-R₅ is deuterium.

In certain embodiments, both R₄ and R₅ are deuterium.

In certain embodiments at least one of R₁-R₃ is deuterium.

In certain embodiments, at least two of R₁-R₃ are deuterium.

In certain embodiments, all of R₁-R₃ are deuterium.

One or more embodiments provide a compound or intermediate of Formula V:

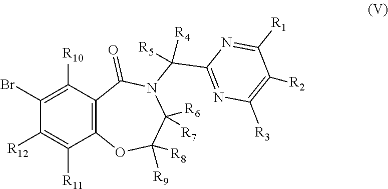

or a pharmaceutically acceptable salt, ester, prodrug, or solvate thereof, wherein:

R₁-R₁₂ are independently selected from hydrogen and deuterium; and at least one R₁-R₁₂ is deuterium, with the proviso that when all of R₆, R₇, R₈, and R₉ are deuterium, at least one of R₁-R₅ or R₁₀-R₁₆ are also deuterium.

Compounds of Formula III may be prepared as shown in the following reaction schemes and the description thereof, as well as relevant literature procedures that may be used by one skilled in the art. Exemplary reagents and procedures for these reactions appear hereinafter and in the working Examples. Any position shown as hydrogen may optionally be replaced with deuterium.

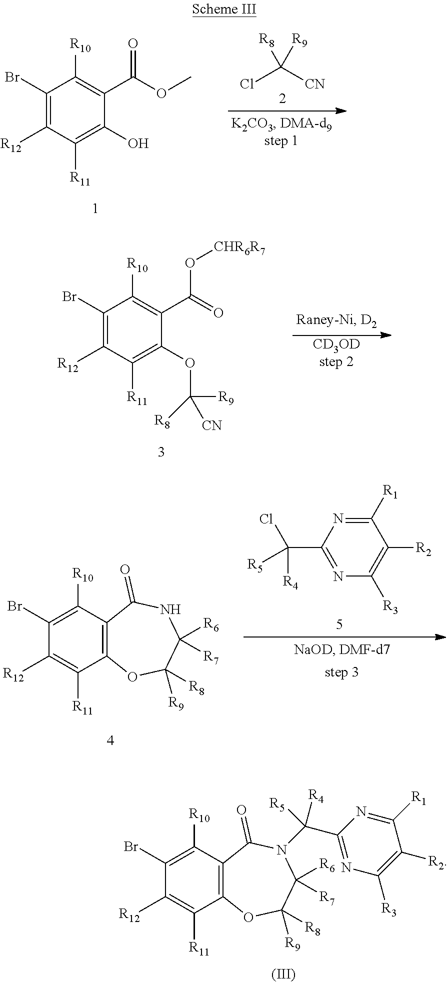

Deuterium can be incorporated into different positions synthetically, according to the synthetic procedures as shown in Scheme III, by using appropriate deuterated intermediates. For example, to introduce deuterium at one or more positions of R₆-R₁₂, compound 1 can be reacted with compound 2 in deuterated DMA (D₂O) to give compound 3, having one or more deuterium atoms at R₆-R₁₂.

The invention is now described with reference to the following examples. Before describing several exemplary embodiments of the invention, it is to be understood that the invention is not limited to the details of construction or process steps set forth in the following description. The invention is capable of other embodiments and of being practiced or being carried out in various ways.

The following abbreviations may be employed in the Examples and elsewhere herein:

DMA=dimethylacetamide

DMF=dimethylformamide

DMSO=dimethyl sulfoxide

DCM=dichloromethane

L=liter

mL=milliliter

μL-microliter

g=gram(s)

mg=milligram(s)

mol=moles

mmol=millimole(s)

h or hr=hour(s)

min=minute(s)

Equiv=equivalent(s)

H₂=hydrogen

Ar=argon

N₂=nitrogen

RT or R.T.=room temperature

AT=ambient temperature

Aq.=aqueous

HPLC=high performance liquid chromatography

HPLC R,=HPLC retention time

LC/MS=high performance liquid chromatography/mass spectrometry

MS or Mass Spec=mass spectrometry

NMR=nuclear magnetic resonance

NMR spectral data: s=singlet; d=doublet; m=multiplet; br=broad; t=triplet

mp=melting point

All IUPAC names were generated using PerkinElmer®'s ChemDraw.

### EXAMPLES

### Example 1—Comparative

### 4-(pyrimidin-2-ylmethyl)-7-[4-(trifluoromethoxy)phenyl]-2,3,4,5-tetrahydro-1,4-benzoxazepin-5-one [Eleclazine]

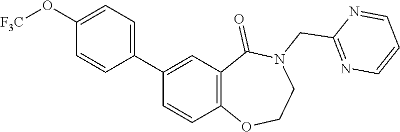 [SMILES: O=C1c2cc(-c3ccc(OC(F)(F)F)cc3)ccc2OCCN1Cc1ncccn1]

### Step 1: Methyl 5-bromo-2-(cyanomethoxy)benzoate

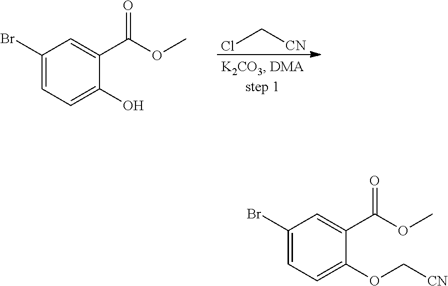

To a solution of 5-bromo-2-hydroxybenzoate (10 g, 43.28 mmol, 1.00 equiv) in DMA (100 mL) was added potassium carbonate (9 g, 65.12 mmol, 1.50 equiv) and 2-chloroacetonitrile (3.4 mL, 1.25 equiv). The resulting suspension was stirred overnight. The solids were filtered out. The filtrate was washed with water. The resulting solution was extracted with ethyl acetate (3×50 mL). The organic layers were dried over anhydrous sodium sulfate and concentrated under vacuum to afford 11 g (94%) of methyl 5-bromo-2-(cyanomethoxy)benzoate as a white solid. LC-MS: m/z=270 [M+H]⁺.

### Step 2: 7-bromo-2,3,4,5-tetrahydro-1,4-benzoxazepin-5-one

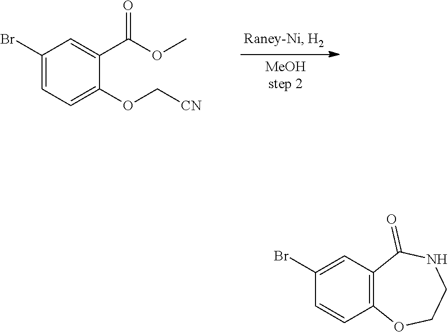

To a solution of 5-bromo-2-(cyanomethoxy)benzoate [Example 1, Step 1] (4 g, 14.81 mmol, 1.00 equiv) in methanol (50 mL) was added saturated aq. NH₃ (4 mL) and Raney-Ni (2 mL) under a H₂ atmosphere. The resulting solution was stirred overnight at room temperature. The catalyst was filtered out. The filtrate was concentrated under vacuum. The residue was purified by SiO₂ chromatography eluted with ethyl acetate/petroleum ether (1:1) to afford 530 mg (15%) of 7-bromo-2,3,4,5-tetrahydro-1,4-benzoxazepin-5-one as a yellow solid. LC-MS: m/z=242 [M+H]⁺.

### Step 3: 7-bromo-4-(pyrimidin-2-ylmethyl)-2,3,4,5-tetrahydro-1,4-benzoxazepin-5-one

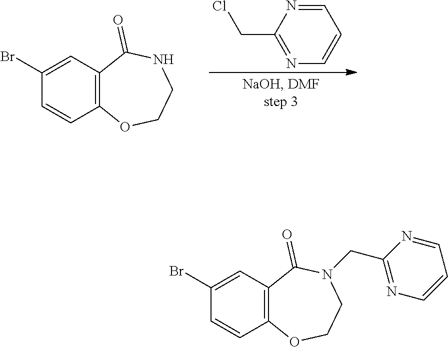 [SMILES: O=C1NCCOc2ccc(Br)cc21 ; ClCc1ncccn1 ; O=C1c2cc(Br)ccc2OCCN1Cc1ncccn1]

To a solution of 7-bromo-2,3,4,5-tetrahydro-1,4-benzoxazepin-5-one [Example 1, Step 2] (530 mg, 2.19 mmol, 1.00 equiv) and 2-(chloromethyl)pyrimidine hydrochloride (650 mg, 3.96 mmol, 1.80 equiv) in DMF (10 mL), was slowly added a NaOH solution (0.55 mL, 10 M, 2.50 equiv), which was stirred at room temperature for 10 min. Then the mixture was stirred at 95° C. for 2 h. After cooling the reaction mixture, ethyl acetate (30 mL) was added and the organic layer was separated. The organic layers were washed with water, brine, dried over anhydrous sodium sulfate, and concentrated under vacuum to afford 600 mg (82%) of 7-bromo-4-(pyrimidin-2-ylmethyl)-2,3,4,5-tetrahydro-1,4-benzoxazepin-5-one as light yellow oil. LC-MS: m/z=334 [M+H]⁺.

### Step 4: 4-(pyrimidin-2-ylmethyl)-7-[4-(trifluoromethoxy)phenyl]-2,3,4,5-tetrahydro-1,4-benzoxazepin-5-one

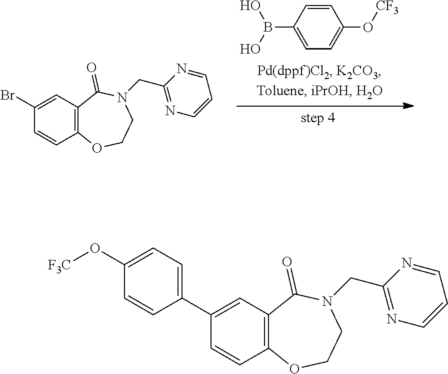 [SMILES: OB(O)c1ccc(OC(F)(F)F)cc1 ; O=C1c2cc(Br)ccc2OCCN1Cc1ncccn1 ; O=C1c2cc(-c3ccc(OC(F)(F)F)cc3)ccc2OCCN1Cc1ncccn1]

To a solution of 7-bromo-4-(pyrimidin-2-ylmethyl)-2,3,4,5-tetrahydro-1,4-benzoxaze-pin-5-one [Example 1, Step 3] (277 mg, 0.83 mmol, 1.00 equiv) in Toluene/iPrOH/H₂O (2:1:1, 4 mL) was added potassium carbonate (459 mg, 3.32 mmol, 4.00 equiv) and [4-(trifluoromethoxy)phenyl]boronic acid (257 mg, 1.25 mmol, 1.50 equiv). The mixture was stirred for 10 min at room temperature. Then Pd(dppf)Cl₂ (12 mg, 0.02 equiv) was added to the solution. The mixture was stirred at 85° C. for 2 h. After cooling the reaction mixture, ethyl acetate (30 mL) was added, and the organic layer was separated. The organic layer was washed with water, brine, dried over anhydrous sodium sulfate, and concentrated under vacuum. The crude product was purified by Prep-HPLC with the following conditions: Column,)(Bridge Prep C18 OBD Column, 5 um, 19*150 mm; mobile phase, Water (10 mmol/L NH₄HCO₃) and CH₃CN (50.0% CH₃CN up to 52.0% in 7 min); Detector, UV 254, 220 nm to afford 190 mg (55%) of 4-(pyrimidin-2-ylmethyl)-7-[4-(trifluoromethoxy)phenyl]-2,3,4,5-tetrahydro-1,4-benzoxazepin-5-one as a white solid. LC-MS: m/z=416 [M+H]⁺

¹H NMR (400 MHz, Chloroform-d) δ 8.75-8.74 (m, 2H), 8.20-8.19 (m, 1H), 7.66-7.61 (m, 3H), 7.29-7.28 (m, 1H), 7.27-7.26 (m, 1H), 7.24-7.23 (m, 1H), 7.13-7.11 (m, 1H), 5.12 (s, 2H), 4.60-4.57 (m, 2H), 3.81-3.78 (m, 2H).

### Example 2: 4-(pyrimidin-2-ylmethyl-d₂)-7-(4-(trifluoromethoxy)phenyl)-3,4-dihydrobenzo[f][1,4]oxazepin-5(2H)-one

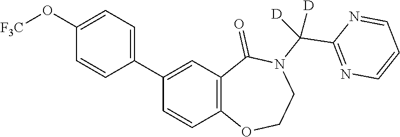 [SMILES: [2H]C([2H])(c1ncccn1)N1CCOc2ccc(-c3ccc(OC(F)(F)F)cc3)cc2C1=O]

### Step 1: pyrimidin-2-ylmethan-d₂-ol

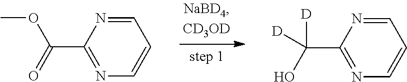

To a stirred solution of methyl pyrimidine-2-carboxylate (5 g, 36.20 mmol, 1.00 equiv) in CD₃OD (50 mL), cooled to 0° C., was added NaBD₄ (1.52 g, 1.00 equiv). The solution was stirred for 2 h. After consumption of the starting material, the reaction was quenched with cold water and concentrated under vacuum to give the crude material which was purified by silica gel column chromatography to afford 3 g (74%) of pyrimidin-2-ylmethan-d₂-ol as white oil. LC-MS: m/z=113 [M+H]⁺.

### Step 2: 2-(chloromethyl-d₂)pyrimidine

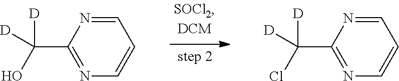 [SMILES: [2H]C([2H])(O)c1ncccn1 ; [2H]C([2H])(Cl)c1ncccn1]

To a stirred solution of pyrimidin-2-ylmethan-d₂-ol [Example 2, Step 1] (3 g, 26.76 mmol, 1.00 equiv) in DCM (40 mL) was added SOCl₂ (9.48 g, 3.00 equiv) at 0° C. under inert atmosphere. The reaction mixture was heated up to 50° C. and stirred for 2 h. The mixture was evaporated under reduced pressure. The residue was quenched with ice cold water followed by saturated NaHCO₃ and extracted with DCM. The organic layers were dried over anhydrous sodium sulfate and concentrated under vacuum to afford 2 g (57%) of 2-(chloromethyl-d₂)pyrimidine as brown oil. LC-MS: m/z=131 [M+H]⁺.

### Step 3: 7-bromo-4-[pyrimidin-2-yl(d₂)methyl]-2,3,4,5-tetrahydro-1,4-benzoxazepin-5-one

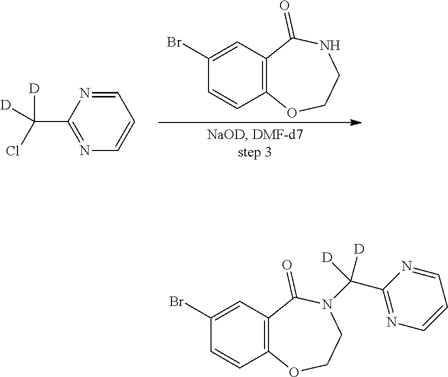 [SMILES: [2H]C([2H])(Cl)c1ncccn1 ; O=C1NCCOc2ccc(Br)cc21 ; [2H]C([2H])(c1ncccn1)N1CCOc2ccc(Br)cc2C1=O]

To a solution of 7-bromo-2,3,4,5-tetrahydro-1,4-benzoxazepin-5-one (560 mg, 2.31 mmol, 1.00 equiv) and 2-(chloromethyl-d₂)pyrimidine [Example 2, Step 2] (544 mg, 4.17 mmol, 1.80 equiv) in DMF-d₇ (10 mL), NaOD solution (1.9 mL, 2.50 equiv) was slowly added and stirred at room temperature for 10 min. Then the mixture was stirred at 95° C. for 2 h. After cooling the reaction mixture, ethyl acetate (30 mL) was added, and the organic layer was separated. The organic layers were washed with water, brine, dried over anhydrous sodium sulfate and concentrated under vacuum to afford 500 mg (64%) of 7-bromo-4-[pyrimidin-2-yl(d₂)methyl]-2,3,4,5-tetrahydro-1,4-benzoxazepin-5-one as yellow oil. LC-MS: m/z=336 [M+H]⁺.

### Step 4: 4-[pyrimidin-2-yl(d₂)methyl]-7-[4-(trifluoromethoxy)phenyl]-2,3,4,5-tetrahydro-1,4-benzoxazepin-5-one

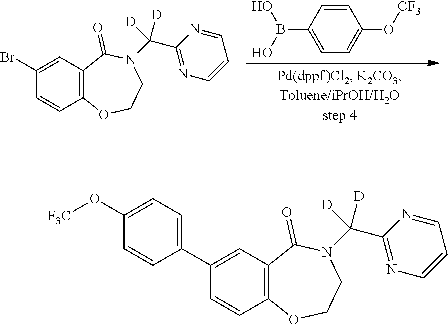 [SMILES: [2H]C([2H])(c1ncccn1)N1CCOc2ccc(Br)cc2C1=O ; OB(O)c1ccc(OC(F)(F)F)cc1 ; [2H]C([2H])(c1ncccn1)N1CCOc2ccc(-c3ccc(OC(F)(F)F)cc3)cc2C1=O]

To a solution of 7-bromo-4-[pyrimidin-2-yl(d₂)methyl]-2,3,4,5-tetrahydro-1,4-benzoxazepin-5-one [Example 2, Step 3] (300 mg, 0.89 mmol, 1.00 equiv) in Toluene/iPrOH/H₂O (2:1:1, 4 mL) was added potassium carbonate (494 mg, 3.57 mmol, 4.00 equiv) and [4-(trifluoromethoxy)phenyl]boronic acid (277 mg, 1.35 mmol, 1.50 equiv). The mixture was stirred for 10 min at room temperature. Then Pd(dppf)Cl₂ (13 mg, 0.02 mmol, 0.02 equiv) was added to the solution. The mixture was stirred at 85° C. for 2 h. After cooling the reaction mixture, ethyl acetate (30 mL) was added, and the organic layer was separated. The organic layer was washed with water, brine, dried over anhydrous sodium sulfate, and concentrated under vacuum. The crude product was purified by Prep-HPLC with the following conditions: Column,)(Bridge Prep C18 OBD Column, 5 um, 19*150 mm; mobile phase, Water (10 mmol/L NH₄HCO₃) and ACN (50.0% ACN up to 52.0% in 7 min); Detector, UV 254, 220 nm. This resulted in 200 mg (54%) of 4-[pyrimidin-2-yl(d₂)methyl]-7-[4-(trifluoromethoxy)phenyl]-2,3,4,5-tetrahydro-1,4-benzoxazepin-5-one as a white solid. LC-MS: m/z=418 [M+H]⁺

¹H NMR (400 MHz, Chloroform-d) δ 8.74-8.73 (m, 2H), 8.20-8.19 (m, 1H), 7.66-7.61 (m, 3H), 7.29-7.28 (m, 1H), 7.27-7.26 (m, 1H), 7.25-7.23 (m, 1H), 7.13-7.11 (m, 1H), 4.59-4.57 (m, 2H), 3.80-3.78 (m, 2H).

### Example 3: 4-(pyrimidin-2-ylmethyl)-7-[4-(trifluoromethoxy)phenyl]-2,3,4,5-tetrahydro(2,2-d₂)-1,4-benzoxazepin-5-one

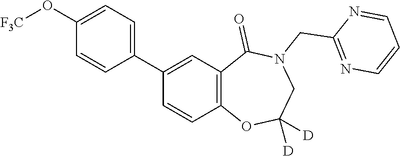 [SMILES: [2H]C1([2H])CN(Cc2ncccn2)C(=O)c2cc(-c3ccc(OC(F)(F)F)cc3)ccc2O1]

### Step 1: [(benzylamino)methyl](d₂)methanol

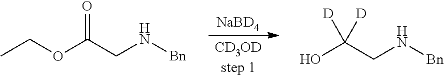

To a solution of ethyl 2-(benzylamino)acetate (10 g, 51.75 mmol, 1.00 equiv) in CD₃OD (50 mL) was slowly added NaBD₄ (6.53 g, 3.00 equiv). Then the mixture was stirred at room temperature overnight. The reaction was quenched by the addition of D₂O (20 mL). The resulting solution was extracted with DCM (3×50 mL). The organic layers were dried over anhydrous sodium sulfate and concentrated under vacuum. The crude product was purified by SiO₂ chromatography eluted with DCM/MeOH (5:1) to afford 6 g (76%) of [(benzylamino)methyl](d₂)methanol as light yellow oil. LC-MS: m/z=154 [M+H]⁺.

### Step 2: N-benzyl-5-bromo-2-fluoro-N-[2-hydroxy(2,2-d₂)ethyl]benzamide

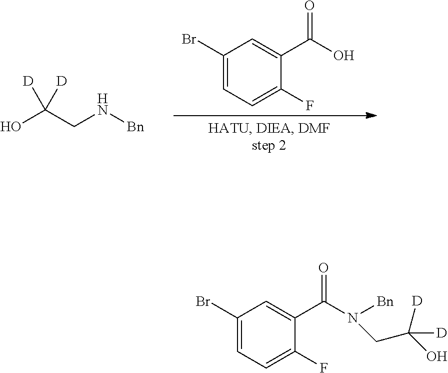 [SMILES: [2H]C([2H])(O)CNCc1ccccc1 ; O=C(O)c1cc(Br)ccc1F ; [2H]C([2H])(O)CN(Cc1ccccc1)C(=O)c1cc(Br)ccc1F]

To a solution of 5-bromo-2-fluorobenzoic acid (6.3 g, 28.77 mmol, 1.00 equiv), [(benzylamino)methyl](d₂)methanol [Example 3, Step 1] (4 g, 26.11 mmol, 0.90 equiv), and DIEA (11.2 g, 86.66 mmol, 3.00 equiv) in dry DMF (100 mL) was added HATU (16.5 g, 43.39 mmol, 1.50 equiv). The mixture was stirred at room temperature overnight. Then to this reaction mixture was added ethyl acetate (50 mL). The solution was washed with brine, dried over anhydrous sodium sulfate, and concentrated under vacuum. The crude product was purified by SiO₂ chromatography eluted with petroleum ether/ethyl acetate (2:3) to afford 6 g (59%) of N-benzyl-5-bromo-2-fluoro-N-[2-hydroxy(2,2-d₂)ethyl] benzamide as light yellow oil. LC-MS: m/z=354 [M+H]⁺.

### Step 3: 4-benzyl-7-bromo-2,3,4,5-tetrahydro(2,2-d₂)-1,4-benzoxazepin-5-one

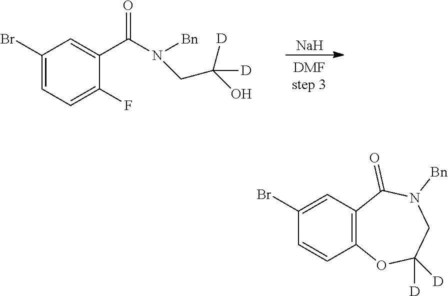

To a solution of N-benzyl-5-bromo-2-fluoro-N-[2-hydroxy(2,2-d₂)ethyl]benzamide [Example 3, Step 2] (7.8 g, 22.02 mmol, 1.00 equiv) in DMF (50 mL) was added sodium hydride (1.15 g, 1.30 equiv). The mixture was stirred at room temperature for 2 h. The reaction was quenched by the addition of H₂O (10 mL). The resulting solution was extracted with ethyl acetate (3×50 mL). The organic layers were dried over anhydrous sodium sulfate and concentrated under vacuum. The crude product was purified by SiO₂ chromatography eluted with petroleum ether/ethyl acetate (9:1) to afford 4 g (54%) of 4-benzyl-7-bromo-2,3,4,5-tetrahydro(2,2-d₂)-1,4-benzoxazepin-5-one as light yellow oil. LC-MS: m/z=334 [M+H]⁺.

### Step 4: 4-benzyl-7-[4-(trifluoromethoxy)phenyl]-2,3,4,5-tetrahydro(2,2-d₂)-1,4-benzoxazepin-5-one

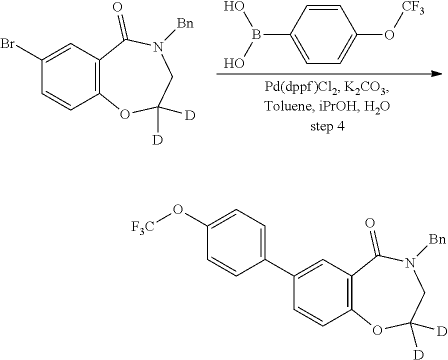 [SMILES: [2H]C1([2H])CN(Cc2ccccc2)C(=O)c2cc(Br)ccc2O1 ; OB(O)c1ccc(OC(F)(F)F)cc1 ; [2H]C1([2H])CN(Cc2ccccc2)C(=O)c2cc(-c3ccc(OC(F)(F)F)cc3)ccc2O1]

To a solution of 4-benzyl-7-bromo-2,3,4,5-tetrahydro(2,2-d₂)-1,4-benzoxazepin-5-one [Example 3, Step 3] (1.5 g, 4.49 mmol, 1.00 equiv) and [4-(trifluoromethoxy)phenyl]boronic acid (1.4 g, 6.80 mmol, 1.50 equiv) in Toluene/iPrOH/H₂O (2:1:1, 24 mL) was added potassium carbonate (2.5 g, 18.09 mmol, 4.00 equiv). The mixture was stirred at room temperature for 10 min. Then Pd(dppf)Cl₂ (66 mg, 0.02 equiv) was added. The resulting solution was stirred for 2 h at 85° C. The reaction was diluted with ethyl acetate (50 mL). The organic layers were dried over anhydrous sodium sulfate and concentrated under vacuum. The crude product was purified by SiO₂ chromatography eluted with petroleum ether/ethyl acetate (9:1) to afford 1.2 g (64%) of 4-benzyl-7-[4-(trifluoromethoxy)phenyl]-2,3,4,5-tetrahydro(2,2-d₂)-1,4-benzoxazepin-5-one as a light yellow solid. LC-MS: m/z=416 [M+H]⁺.

### Step 5: 7-[4-(trifluoromethoxy)phenyl]-2,3,4,5-tetrahydro(2,2-d₂)-1,4-benzoxazepin-5-one

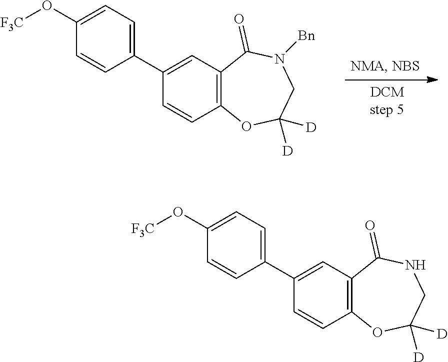 [SMILES: [2H]C1([2H])CN(Cc2ccccc2)C(=O)c2cc(-c3ccc(OC(F)(F)F)cc3)ccc2O1 ; [2H]C1([2H])CNC(=O)c2cc(-c3ccc(OC(F)(F)F)cc3)ccc2O1]

To a solution of 4-benzyl-7-[4-(trifluoromethoxy)phenyl]-2,3,4,5-tetrahydro(2,2-d₂)-1,4-benzoxazepin-5-one [Example 3, Step 4] (400 mg, 0.96 mmol, 1.00 equiv) in DCM (8 mL) was added NBS (429 mg, 2.41 mmol, 2.50 equiv) and NMA (14 mg, 0.20 equiv). The resulting solution was stirred for 48 h at 50° C. The mixture was washed with H₂O (3×20 mL). The organic layers were dried over anhydrous sodium sulfate and concentrated under vacuum. The crude product was purified by SiO₂ chromatography eluted with petroleum ether/ethyl acetate (3:1) to afford 80 mg (26%) of 7-[4-(trifluoromethoxy)phenyl]-2,3,4,5-tetrahydro(2,2-d₂)-1,4-benzoxazepin-5-one as a light yellow solid. LC-MS: m/z=326 [M+H]⁺.

### Step 6: 4-(pyrimidin-2-ylmethyl)-7-[4-(trifluoromethoxy)phenyl]-2,3,4,5-tetrahydro(2,2-d₂)-1,4-benzoxazepin-5-one

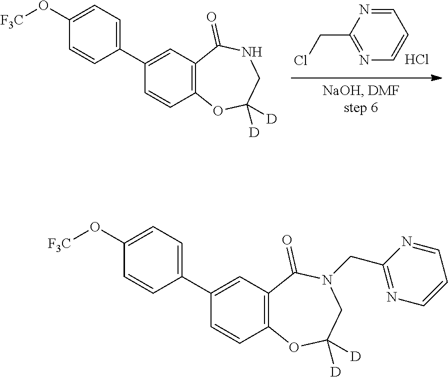 [SMILES: [2H]C1([2H])CNC(=O)c2cc(-c3ccc(OC(F)(F)F)cc3)ccc2O1 ; ClCc1ncccn1 ; Cl ; [2H]C1([2H])CN(Cc2ncccn2)C(=O)c2cc(-c3ccc(OC(F)(F)F)cc3)ccc2O1]

To a solution of 7-[4-(trifluoromethoxy)phenyl]-2,3,4,5-tetrahydro(2,2-d₂)-1,4-benzoxazepin-5-one [Example 3, Step 5] (320 mg, 0.98 mmol, 1.00 equiv) and 2-(chloromethyl) pyrimidine hydrochloride (323 mg, 1.96 mmol, 2.00 equiv) in DMF (8 mL), was slowly added a NaOH solution (0.24 mL, 10 M, 2.50 equiv). The solution was stirred at room temperature for 10 min. Then the mixture was stirred at 95° C. for 4 h. After cooling the reaction mixture, ethyl acetate (30 mL) was added, and the organic layer was separated. The organic layers were washed with water, dried over anhydrous sodium sulfate, and concentrated under vacuum. The crude product was purified by Prep-HPLC with the following conditions: Column,)(Bridge Prep OBD C18 Column, 30*150 mm 5 um; mobile phase, Water (0.05% NH₃H₂O) and CH₃CN (50.0% CH₃CN up to 55.0% in 7 min); Detector, UV 254, 220 nm. This resulted in 150 mg (37%) of 4-(pyrimidin-2-ylmethyl)-7-[4-(trifluoromethoxy)phenyl]-2,3,4,5-tetrahydro(2,2-d₂)-1,4-benzoxazepin-5-one as a white solid. LC-MS: m/z=418 [M+H]⁺

¹H NMR (400 MHz, Chloroform-d) δ 8.76-8.74 (m, 2H), 8.21-8.20 (m, 1H), 7.66-7.61 (m, 3H), 7.30-7.29 (m, 1H), 7.28-7.26 (m, 1H), 7.25-7.24 (m, 1H), 7.14-7.11 (m, 1H), 5.13 (s, 2H), 3.79 (s, 2H).

### Example 4: 4-(pyrimidin-2-ylmethyl)-7-(4-(trifluoromethoxy)phenyl)-3,4-dihydrobenzo[f][1,4]oxazepin-5(2H)-one-3,3-d₂

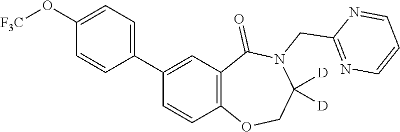 [SMILES: [2H]C1([2H])COc2ccc(-c3ccc(OC(F)(F)F)cc3)cc2C(=O)N1Cc1ncccn1]

### Step 1: methyl 2-amino(d₂)acetate

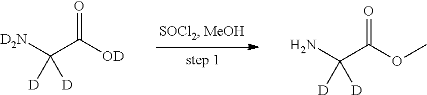

To a solution of 2-[(d₂)amino](d₂)ethan(d)oic acid (10 g, 124.85 mmol, 1.00 equiv) in methanol (100 mL) was added thionyl chloride (36.9 g, 2.50 equiv) dropwise at 0° C. The reaction was stirred at 65° C. for 4 h. Then the mixture was concentrated in vacuo to afford 11 g (97%) of methyl 2-amino(d₂)acetate as a white solid.

¹H NMR (400 MHz, Deuterium Oxide) δ 3.77 (s, 3H).

### Step 2: methyl 2-(benzylamino)(d₂)acetate

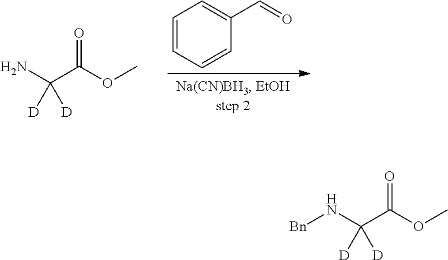

To a solution of methyl 2-amino(d₂)acetate [Example 4, Step 1] (11 g, 120.74 mmol, 1.00 equiv) in ethanol (150 mL) was added Na(CN)BH₃ (11.4 g, 1.50 equiv). Benzaldehyde (8.97 g, 84.53 mmol, 0.70 equiv) was added to this mixture dropwise. The reaction was stirred at room temperature overnight. Then the solution was concentrated to remove EtOH. The resulting solution was extracted with DCM (3×50 mL). The organic layers were dried over anhydrous sodium sulfate and concentrated under vacuum. The crude product was purified by SiO₂ chromatography eluted with petroleum ether/ethyl acetate (5:1) to afford 7.5 g (34%) of methyl 2-(benzylamino)(d₂)acetate as colorless oil.

### Step 3: 2-(benzylamino)(2,2-d₂)ethan-1-ol

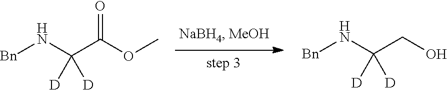

To a solution of methyl 2-(benzylamino)(d₂)acetate [Example 4, Step 2] (7.5 g, 41.38 mmol, 1.00 equiv) in methanol (100 mL) was slowly added NaBH₄ (4.72 g, 124.77 mmol, 3.00 equiv). Then the mixture was stirred at room temperature overnight. The reaction was quenched by the addition of H₂O (10 mL). The resulting solution was extracted with DCM (3×50 mL). The organic layers were dried over anhydrous sodium sulfate and concentrated under vacuum. This resulted in 5 g (79%) of 2-(benzylamino)(2,2-d₂)ethan-1-ol as colorless oil.

### Step 4: N-benzyl-5-bromo-2-fluoro-N-[2-hydroxy(1,1-d₂)ethyl]benzamide

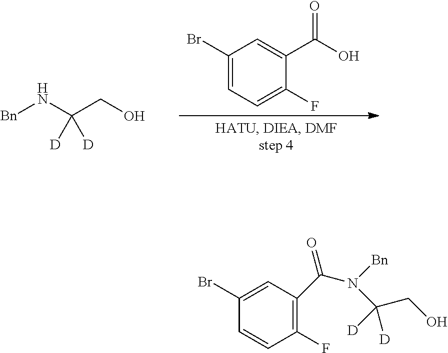 [SMILES: [2H]C([2H])(CO)NCc1ccccc1 ; O=C(O)c1cc(Br)ccc1F ; [2H]C([2H])(CO)N(Cc1ccccc1)C(=O)c1cc(Br)ccc1F]

To a solution of 5-bromo-2-fluorobenzoic acid (6.59 g, 30.09 mmol, 1.00 equiv), 2-(benzylamino)(2,2-d₂)ethan-1-ol [Example 4, Step 3] (4.18 g, 27.28 mmol, 0.90 equiv), and DIEA (11.7 g, 90.53 mmol, 3.00 equiv) in dry DMF (80 mL) was added HATU (17.23 g, 45.31 mmol, 1.50 equiv). The mixture was stirred at room temperature overnight. Then, to this reaction mixture, was added ethyl acetate (80 mL). The solution was washed with brine, dried over anhydrous sodium sulfate, and concentrated under vacuum. The crude product was purified by SiO₂ chromatography eluted with petroleum ether/ethyl acetate (2:3) to afford 7 g (66%) of N-benzyl-5-bromo-2-fluoro-N-[2-hydroxy(1,1-d₂)ethyl]benzamide as light yellow oil.

### Step 5: 4-benzyl-7-bromo-2,3,4,5-tetrahydro(3,3-d₂)-1,4-benzoxazepin-5-one

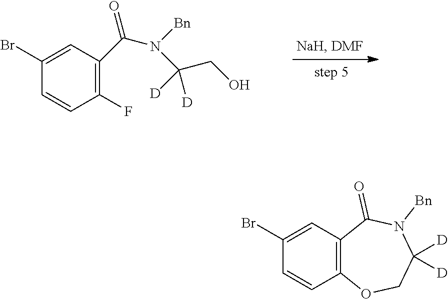 [SMILES: [2H]C([2H])(CO)N(Cc1ccccc1)C(=O)c1cc(Br)ccc1F ; [2H]C1([2H])COc2ccc(Br)cc2C(=O)N1Cc1ccccc1]

To a solution of N-benzyl-5-bromo-2-fluoro-N-[2-hydroxy(1,1-d₂)ethyl]benzamide [Example 4, Step 4] (7 g, 19.76 mmol, 1.00 equiv) in DMF (100 mL) was added sodium hydride (1.03 g, 1.30 equiv). The mixture was stirred at room temperature for 4 h. Then the reaction was quenched by the addition of H₂O (10 mL). The resulting solution was extracted with ethyl acetate (3×50 mL). The organic layers were dried over anhydrous sodium sulfate and concentrated under vacuum. The crude product was purified by SiO₂ chromatography eluted with petroleum ether/ethyl acetate (9:1) to afford 4 g (61%) of 4-benzyl-7-bromo-2,3,4,5-tetrahydro(3,3-d₂)-1,4-benzoxazepin-5-one as colorless oil.

### Step 6: 4-benzyl-7-[4-(trifluoromethoxy)phenyl]-2,3,4,5-tetrahydro(3,3-d₂)-1,4-benzoxazepin-5-one

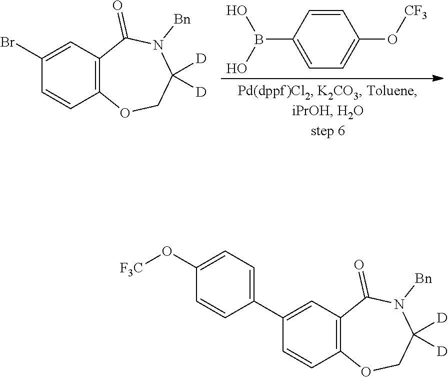

To a solution of 4-benzyl-7-bromo-2,3,4,5-tetrahydro(3,3-d₂)-1,4-benzoxazepin-5-one [Example 4, Step 5] (3 g, 8.98 mmol, 1.00 equiv) and [4-(trifluoromethoxy)phenyl]boronic acid (2.78 g, 13.50 mmol, 1.50 equiv) in Toluene/iPrOH/H₂O (2:1:1, 28 mL) was added potassium carbonate (4.97 g, 35.96 mmol, 4.00 equiv). The mixture was stirred at room temperature for 10 min. Then Pd(dppf)Cl₂ (132 mg, 0.18 mmol, 0.02 equiv) was added. The resulting solution was stirred for 2 h at 85° C. The reaction was diluted with ethyl acetate (50 mL). The organic layers were dried over anhydrous sodium sulfate and concentrated under vacuum. The crude product was purified by SiO₂ chromatography eluted with petroleum ether/ethyl acetate (9:1) to afford 3 g (80%) of 4-benzyl-7-[4-(trifluoromethoxy)phenyl]-2,3,4,5-tetrahydro(3,3-d₂)-1,4-benzoxazepin-5-one as a light yellow solid.

### Step 7: 7-[4-(trifluoromethoxy)phenyl]-2,3,4,5-tetrahydro(3,3-d₂)-1,4-benzoxazepin-5-one

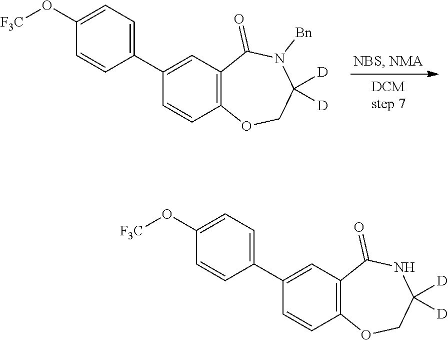 [SMILES: [2H]C1([2H])COc2ccc(-c3ccc(OC(F)(F)F)cc3)cc2C(=O)N1Cc1ccccc1 ; [2H]C1([2H])COc2ccc(-c3ccc(OC(F)(F)F)cc3)cc2C(=O)N1]

To a solution of 4-benzyl-7-[4-(trifluoromethoxy)phenyl]-2,3,4,5-tetrahydro(3,3-d₂)-1,4-benzoxazepin-5-one [Example 4, Step 6] (2 g, 4.81 mmol, 1.00 equiv) in DCM (30 mL) was added NBS (2.14 g, 12.02 mmol, 2.50 equiv) and NMA (70 mg, 0.20 equiv). The resulting solution was stirred for 48 h at 50° C. The mixture was washed with H₂O (3×20 mL). The organic layers were dried over anhydrous sodium sulfate and concentrated under vacuum. The crude product was purified by SiO₂ chromatography eluted with petroleum ether/ethyl acetate (3:1) to afford 220 mg (14%) of 7-[4-(trifluoromethoxy)phenyl]-2,3,4,5-tetrahydro(3,3-d₂)-1,4-benzoxazepin-5-one as a light yellow solid.

### Step 8: 4-(pyrimidin-2-ylmethyl)-7-[4-(trifluoromethoxy)phenyl]-2,3,4,5-tetrahydro(3,3-d₂)-1,4-benzoxazepin-5-one

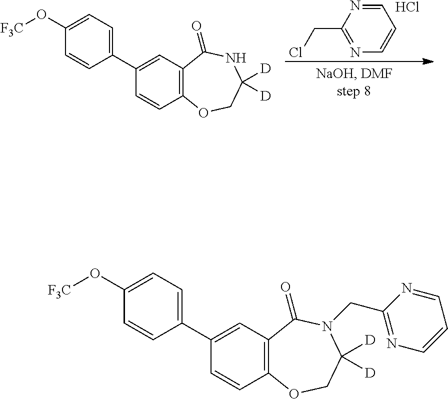

To a solution of 7-[4-(trifluoromethoxy)phenyl]-2,3,4,5-tetrahydro(3,3-d₂)-1,4-benzoxazepin-5-one [Example 4, Step 7] (300 mg, 0.92 mmol, 1.00 equiv) and 2-(chloromethyl) pyrimidine hydrochloride (303 mg, 1.84 mmol, 2.00 equiv) in DMF (8 mL), was slowly added a NaOH solution (0.23 mL, 10 M, 2.50 equiv). The reaction mixture was stirred at room temperature for 10 min. Then the mixture was stirred at 95° C. for 4 h. After cooling the reaction mixture, ethyl acetate (20 mL) was added, and the organic layer was separated. The organic layers were washed with water, dried over anhydrous sodium sulfate, and concentrated under vacuum. The crude product was purified by Prep-HPLC with the following conditions: Column) (Bridge Prep OBD C18 Column, 19*250 mm, 5 um; mobile phase, Water (10 mmol/L NH₄HCO₃) and CH₃CN (62.0% CH₃CN up to 66.0% in 7 min); Detector, UV 220, 254 nm to afford 180 mg (47%) of 4-(pyrimidin-2-ylmethyl)-7-[4-(trifluoromethoxy)phenyl]-2,3,4,5-tetrahydro(3,3-d₂)-1,4-benzoxazepin-5-one as a white solid. LC-MS: m/z=418 [M+H]⁺

¹H NMR (400 MHz, Chloroform-d) δ 8.74-8.73 (m, 2H), 8.20-8.19 (m, 1H), 7.65-7.61 (m, 3H), 7.29-7.27 (m, 1H), 7.24-7.23 (m, 1H), 7.23-7.22 (m, 1H), 7.13-7.11 (m, 1H), 5.12 (s, 2H), 4.57 (s, 2H).

### Example 5: 4-(pyrimidin-2-ylmethyl)-7-[4-(trifluoromethoxy)phenyl]-2,3,4,5-tetrahydro(2,2,3,3-d₄)-1,4-benzoxazepin-5-one

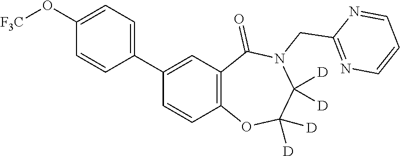 [SMILES: [2H]C1([2H])Oc2ccc(-c3ccc(OC(F)(F)F)cc3)cc2C(=O)N(Cc2ncccn2)C1([2H])[2H]]

### Step 1: 5-bromo-2-hydroxybenzamide

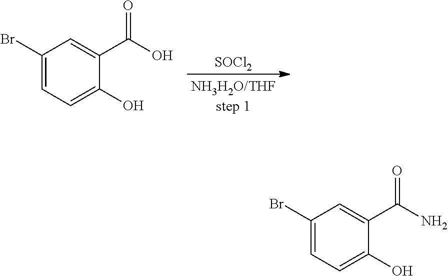

A solution of 5-bromo-2-hydroxybenzoic acid (20 g, 92.16 mmol, 1.00 equiv) in thionyl chloride (50 mL) was stirred at 80° C. for 4 h. The mixture was concentrated under vacuum. Then the residue was added to a solution of ammonia (50 mL) in tetrahydrofuran (50 mL) and stirred at room temperature for 2 h. Ethyl acetate (50 mL) was added to the reaction. The solid was filtered out. The filtrate was concentrated under vacuum and purified by SiO₂ chromatography eluted with ethyl acetate/petroleum ether (1:5) to afford 4.5 g (23%) of 5-bromo-2-hydroxybenzamide as a yellow solid. LC-MS: m/z=216 [M+H]⁺.

### Step 2: 5-bromo-2-[2-bromo(d₄)ethoxy]benzamide

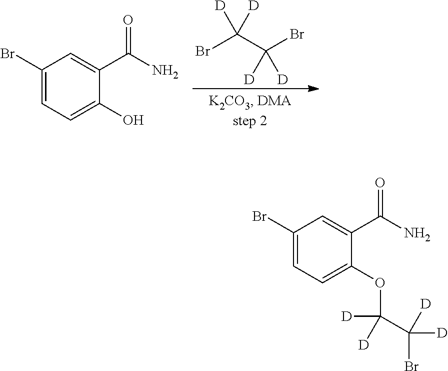 [SMILES: NC(=O)c1cc(Br)ccc1O ; [2H]C([2H])(Br)C([2H])([2H])Br ; [2H]C([2H])(Br)C([2H])([2H])Oc1ccc(Br)cc1C(N)=O]

To a solution of 5-bromo-2-hydroxybenzamide [Example 5, Step 1] (3.2 g, 14.81 mmol, 1.00 equiv) in DMA (25 mL) was added potassium carbonate (6.16 g, 44.57 mmol, 3.00 equiv) and dibromo(d₄)ethane (5.72 g, 29.81 mmol, 2.00 equiv). The resulting solution was stirred for 5 h at room temperature. The reaction mixture was filtered. The filtrate was washed with water (50 mL). The organic layer was concentrated under vacuum to afford 2 g (41%) of 5-bromo-2-[2-bromo(d₄)ethoxy]benzamide as a yellow solid. LC-MS: m/z=326 [M+H]⁺.

### Step 3: 7-bromo-2,3,4,5-tetrahydro(2,2,3,3-d₄)-1,4-benzoxazepin-5-one

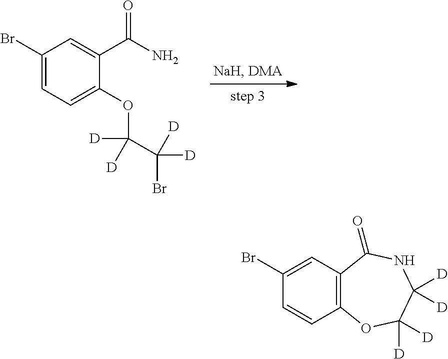 [SMILES: [2H]C([2H])(Br)C([2H])([2H])Oc1ccc(Br)cc1C(N)=O ; [2H]C1([2H])NC(=O)c2cc(Br)ccc2OC1([2H])[2H]]

To a suspension of sodium hydride (350 mg, 1.30 equiv) in DMA (20 mL) was slowly added a solution of 5-bromo-2-[2-bromo(d₄)ethoxy]benzamide [Example 5, Step 2] (2.2 g, 6.73 mmol, 1.00 equiv) in DMA (5 mL). The resulting solution was stirred for 3 h at room temperature. The reaction was quenched by the addition of D₂O (5 mL). The resulting solution was extracted with ethyl acetate (3×50 mL). The organic layers were dried over anhydrous sodium sulfate and concentrated under vacuum. The crude product was purified by SiO₂ chromatography eluted with ethyl acetate/petroleum ether (3:2) to afford 400 mg (24%) of 7-bromo-2,3,4,5-tetrahydro(2,2,3,3-d₄)-1,4-benzoxazepin-5-one as a yellow solid. LC-MS: m/z=246 [M+H]⁺.

### Step 4: 7-bromo-4-(pyrimidin-2-ylmethyl)-2,3,4,5-tetrahydro(2,2,3,3-d₄)-1,4-benzoxazepin-5-one

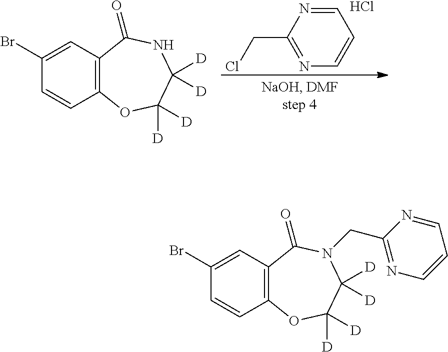

To a solution of 7-bromo-2,3,4,5-tetrahydro(2,2,3,3-d₄)-1,4-benzoxazepin-5-one [Example 5, Step 3] (200 mg, 0.81 mmol, 1.00 equiv) and 2-(chloromethyl)pyrimidine hydrochloride (241 mg, 1.46 mmol, 1.80 equiv) in DMF (5 mL), was slowly added a NaOH solution (0.2 mL, 10M, 2.50 equiv). The reaction mixture was stirred at room temperature for 10 min. Then the mixture was stirred at 95° C. for 2 h. After cooling the reaction mixture, ethyl acetate (20 mL) was added, and the organic layer was separated. The organic layers were washed with water, dried over anhydrous sodium sulfate, and concentrated under vacuum to afford 200 mg (73%) of 7-bromo-4-(pyrimidin-2-ylmethyl)-2,3,4,5-tetrahydro(2,2,3,3-d₄)-1,4-benzoxazepin-5-one as light yellow oil. LC-MS: m/z=338 [M+H]⁺.

### Step 5: 4-(pyrimidin-2-ylmethyl)-7-[4-(trifluoromethoxy)phenyl]-2,3,4,5-tetrahydro(2,2,3,3-d₄)-1,4-benzoxazepin-5-one

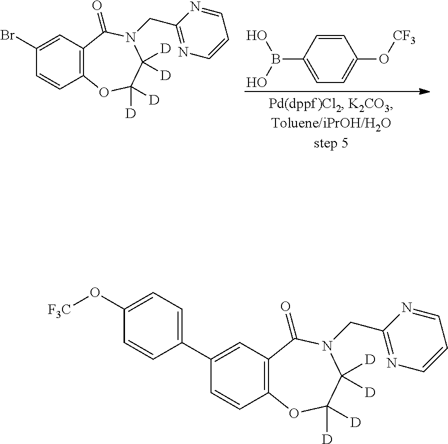

To a solution of 7-bromo-4-(pyrimidin-2-ylmethyl)-2,3,4,5-tetrahydro(2,2,3,3-d₄)-1,4-benzoxazepin-5-one [Example 5, Step 4] (250 mg, 0.74 mmol, 1.00 equiv) in Toluene/iPrOH/H₂O (2:1:1, 8 mL) was added K₂CO₃ (409 mg, 2.96 mmol, 4.00 equiv) and [4-(trifluoromethoxy)phenyl]boronic acid (229 mg, 1.11 mmol, 1.50 equiv). The mixture was stirred for 10 min at room temperature. Then Pd(dppf)Cl₂ (11 mg, 0.02 equiv) was added to the solution. The mixture was stirred at 85° C. for 2 h. After cooling the reaction mixture, ethyl acetate (30 mL) was added, and the organic layer was separated. The organic layer was washed with water, dried over anhydrous sodium sulfate, and concentrated under vacuum. The crude product was purified by Prep-HPLC with the following conditions: Column,)(Bridge Prep C18 OBD Column, 5 um, 19*150 mm; mobile phase, Water (10 mmol/L NH₄HCO₃) and CH₃CN (43.0% CH₃CN up to 53.0% in 8 min); Detector, UV 254, 220 nm to afford 180 mg (58%) of 4-(pyrimidin-2-ylmethyl)-7-[4-(trifluoromethoxy)phenyl]-2,3,4,5-tetrahydro(2,2,3,3-d₄)-1,4-benzoxazepin-5-one as a white solid. LC-MS: m/z=420 [M+H]⁺

¹H NMR (400 MHz, Chloroform-d) δ 8.73-8.71 (m, 2H), 8.18-8.17 (m, 1H), 7.63-7.59 (m, 3H), 7.27-7.25 (m, 1H), 7.22-7.21 (m, 1H), 7.20-7.11 (m, 1H), 7.10-7.09 (m, 1H), 5.10 (s, 2H).

### Example 6: 4-[pyrimidin-2-yl(d₂)methyl]-7-[4-(trifluoromethoxy)phenyl]-2,3,4,5-tetrahydro(2,2,3,3-d₄)-1,4-benzoxazepin-5-one

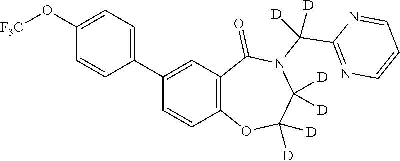 [SMILES: [2H]C([2H])(c1ncccn1)N1C(=O)c2cc(-c3ccc(OC(F)(F)F)cc3)ccc2OC([2H])([2H])C1([2H])[2H]]

### Step 1: 7-bromo-4-[pyrimidin-2-yl(d₂)methyl]-2,3,4,5-tetrahydro(2,2,3,3-d₄)-1,4-benzoxazepin-5-one

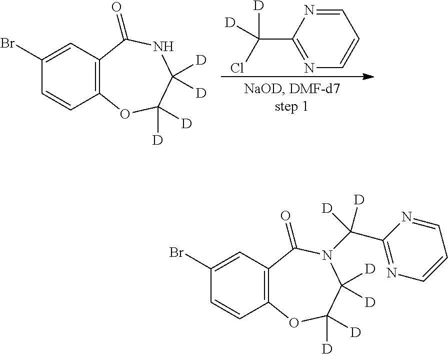

To a solution of 7-bromo-2,3,4,5-tetrahydro(2,2,3,3-d₄)-1,4-benzoxazepin-5-one [Example 5, Step 3] (500 mg, 2.03 mmol, 1.00 equiv) and 2-[chloro(d₂)methyl]pyrimidine (478 mg, 3.66 mmol, 1.80 equiv) in DMF-d7 (7 mL), was slowly added a NaOD solution (0.5 mL, 10 M, 2.50 equiv). The reaction mixture was stirred at room temperature for 10 min. Then the mixture was stirred at 95° C. for 2 h. After cooling the reaction mixture, ethyl acetate (20 mL) was added, and the organic layer was separated. The organic layers were washed with water, dried over anhydrous sodium sulfate and concentrated under vacuum to afford 500 mg (72%) of 7-bromo-4-[pyrimidin-2-yl(d₂)methyl]-2,3,4,5-tetrahydro (2,2,3,3-d₄)-1,4-benzoxazepin-5-one as light yellow oil.

### Step 2: 4-[pyrimidin-2-yl(d₂)methyl]-7-[4-(trifluoromethoxy)phenyl]-2,3,4,5-tetrahydro(2,2,3,3-d₄)-1,4-benzoxazepin-5-one

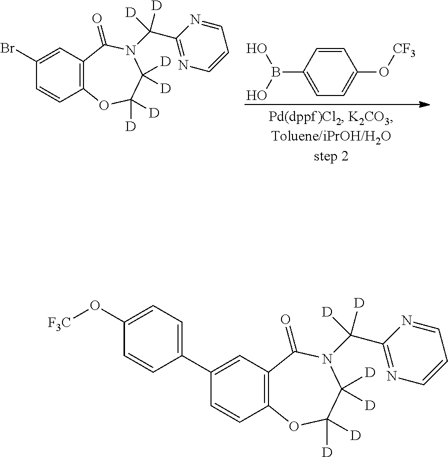

To a solution of 7-bromo-4-[pyrimidin-2-yl(d₂)methyl]-2,3,4,5-tetrahydro(2,2,3,3-d₄)-1,4-benzoxazepin-5-one [Example 6, Step 1] (380 mg, 1.12 mmol, 1.00 equiv) in Toluene/iPrOH/H₂O (2:1:1, 8 mL) was added K₂CO₃ (619 mg, 4.48 mmol, 4.00 equiv) and [4-(trifluoromethoxy)phenyl]boronic acid (346 mg, 1.68 mmol, 1.50 equiv). The mixture was stirred for 10 min at room temperature. Then Pd(dppf)Cl₂ (16 mg, 0.02 equiv) was added to the solution. The mixture was stirred at 85° C. for 2 h. After cooling the reaction mixture, ethyl acetate (30 mL) was added, and the organic layer was separated. The organic layer was washed with water, dried over anhydrous sodium sulfate, and concentrated under vacuum. The crude product was purified by Prep-HPLC with the following conditions: Column,)(Bridge Prep C18 OBD Column, 5 um, 19*150 mm; mobile phase, Water (10 mmol/L NH₄HCO₃) and CH₃CN (40.0% CH₃CN up to 62.0% in 7 min); Detector, UV 254, 220 nm to afford 200 mg (42%) of 4-[pyrimidin-2-yl(d₂)methyl]-7-[4-(trifluoromethoxy)phenyl]-2,3,4,5-tetrahydro(2,2,3,3-d₄)-1,4-benzoxazepin-5-one as a white solid. LC-MS: m/z=422 [M+H]⁺

¹H NMR (400 MHz, Chloroform-d) δ 8.74-8.73 (m, 2H), 8.20-8.19 (m, 1H), 7.65-7.61 (m, 3H), 7.29-7.27 (m, 1H), 7.25-7.23 (m, 1H), 7.23-7.22 (m, 1H), 7.13-7.11 (m, 1H).

### Example 7: 4-(pyrimidin-2-ylmethyl)-7-[4-(trifluoromethoxy)phenyl]-2,3,4,5-tetrahydro(8,9-d₂)-1,4-benzoxazepin-5-one

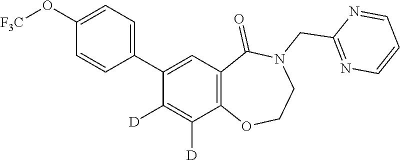 [SMILES: [2H]c1c(-c2ccc(OC(F)(F)F)cc2)cc2c(c1[2H])OCCN(Cc1ncccn1)C2=O]

### Step 1: 2-hydroxy(3,4,5-d₃)benzoic acid

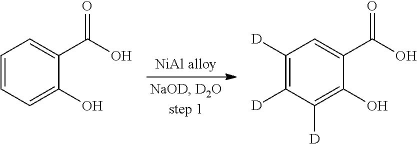 [SMILES: O=C(O)c1ccccc1O ; [2H]c1cc(C(=O)O)c(O)c([2H])c1[2H]]

To a solution of 2-hydroxybenzoic acid (8 g, 57.92 mmol, 1.00 equiv) in D₂O (30 mL) was added NaOD (10 M, 4 mL) and NiAl alloy (800 mg). The resulting solution was stirred for 15 h at 120° C. The pH of the solution was adjusted to 2-3 with HCl (12 M). Then the resulting solution was extracted with ethyl acetate (3×50 mL). The organic layers were dried over anhydrous sodium sulfate and concentrated under vacuum. The progress was repeated three times to afford 6 g (73%) of 2-hydroxy(3,4,5-d₃)benzoic acid as a white solid.

### Step 2: 2-hydroxy(3,4,5-d₃)benzoate

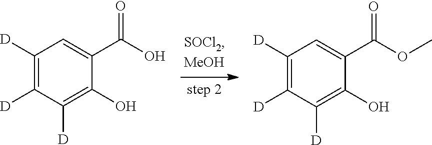

To a solution of 2-hydroxy(3,4,5-d₃)benzoic acid [Example 7, Step 1] (6 g, 42.51 mmol, 1.00 equiv) in methanol (100 mL) was added thionyl chloride (15.06 g, 3.00 equiv). The resulting solution was stirred for 3 h at 65° C. The resulting solution was concentrated under vacuum to afford 5 g (76%) of methyl 2-hydroxy(3,4,5-d₃)benzoate as colorless oil.

### Step 3: methyl 5-bromo-2-hydroxy(3,4-d₂)benzoate

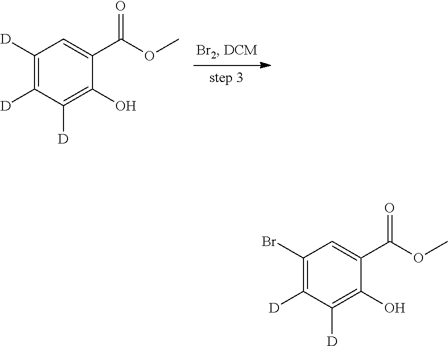

To a solution of methyl 2-hydroxy(3,4,5-d₃)benzoate [Example 7, Step 2] (5 g, 32.22 mmol, 1.00 equiv) in dichloromethane (80 mL) was added Bra (5.57 g, 34.85 mmol, 1.08 equiv) dropwise. The resulting solution was stirred for 5 h at room temperature. Then the resulting solution was washed with the solution of NaHCO₃. The organic layers were dried over anhydrous sodium sulfate and concentrated under vacuum to afford 4 g (53%) of methyl 5-bromo-2-hydroxy(3,4-d₂)benzoate as a white solid.

### Step 4: methyl 5-bromo-2-(cyanomethoxy)(3,4-d₂)benzoate

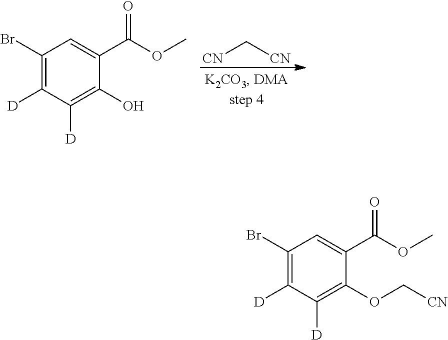

To a solution of methyl 5-bromo-2-hydroxy(3,4-d₂)benzoate [Example 7, Step 3] (4 g, 17.16 mmol, 1.00 equiv) in DMA (50 mL) was added 2-chloroacetonitrile (1.62 g, 21.46 mmol, 1.25 equiv) and potassium carbonate (3.57 g, 25.83 mmol, 1.50 equiv). The resulting suspension was stirred at room temperature overnight. Then the resulting solution was extracted with ethyl acetate (3×50 mL). The organic layers were dried over anhydrous sodium sulfate and concentrated under vacuum to afford 3.7 g (79%) of methyl 5-bromo-2-(cyanomethoxy)(3,4-d₂)benzoate as a white solid.

### Step 5: 7-bromo-2,3,4,5-tetrahydro(8,9-d₂)-1,4-benzoxazepin-5-one

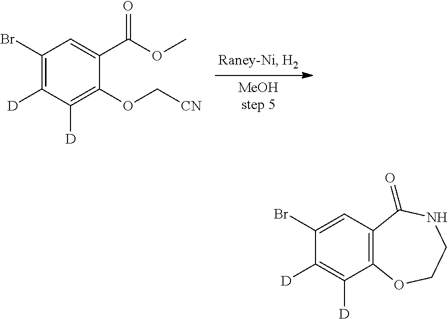 [SMILES: [2H]c1c(Br)cc(C(=O)OC)c(OCC#N)c1[2H] ; [2H]c1c(Br)cc2c(c1[2H])OCCNC2=O]

To a solution of methyl 5-bromo-2-(cyanomethoxy)(3,4-d₂)benzoate [Example 7, Step 4] (3 g, 11.03 mmol, 1.00 equiv) in methanol (50 mL) was added Raney-Ni (500 mg) and NH₃H₂O (3 mL) under a H₂ atmosphere. The resulting solution was stirred overnight at room temperature. The catalyst was filtered out. The filtrate was concentrated under vacuum. The residue was purified by SiO₂ chromatography eluted with ethyl acetate/petroleum acetate (1:1) to afford 300 mg (11%) of 7-bromo-2,3,4,5-tetrahydro(8,9-d₂)-1,4-benzoxazepin-5-one as a white solid.

### Step 6: 7-bromo-4-(pyrimidin-2-ylmethyl)-2,3,4,5-tetrahydro(8,9-d₂)-1,4-benzoxazepin-5-one

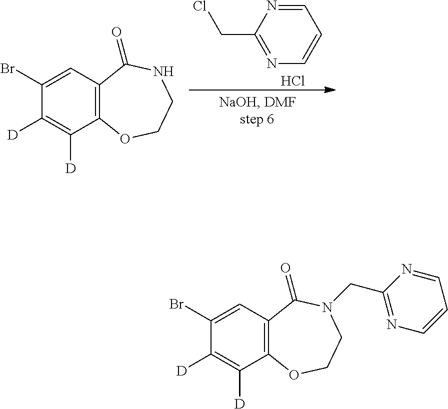 [SMILES: [2H]c1c(Br)cc2c(c1[2H])OCCNC2=O ; ClCc1ncccn1 ; Cl ; [2H]c1c(Br)cc2c(c1[2H])OCCN(Cc1ncccn1)C2=O]

To a solution of 7-bromo-2,3,4,5-tetrahydro(8,9-d₂)-1,4-benzoxazepin-5-one [Example 7, Step 5] (300 mg, 1.23 mmol, 1.00 equiv) and 2-(chloromethyl)pyrimidine hydrochloride (506 mg, 3.07 mmol, 2.50 equiv) in DMF (8 mL), was slowly added a NaOH solution (0.3 mL, 10 M, 2.50 equiv). The reaction mixture was stirred at room temperature for 10 min. Then the mixture was stirred at 95° C. for 2 h. After cooling the reaction mixture, ethyl acetate (30 mL) was added, and the organic layer was separated. The organic layers were washed with water, brine, dried over anhydrous sodium sulfate, and concentrated under vacuum to afford 300 mg (73%) of 7-bromo-4-(pyrimidin-2-ylmethyl)-2,3,4,5-tetrahydro (8,9-d₂)-1,4-benzoxa-zepin-5-one as a light yellow solid.

### Step 7: 4-(pyrimidin-2-ylmethyl)-7-[4-(trifluoromethoxy)phenyl]-2,3,4,5-tetrahydro(8,9-d₂)-1,4-benzoxazepin-5-one

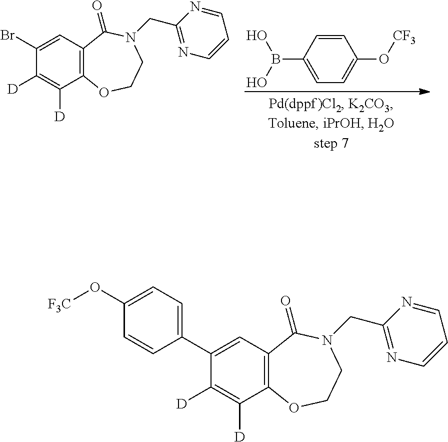

To a solution of 7-bromo-4-(pyrimidin-2-ylmethyl)-2,3,4,5-tetrahydro(8,9-d₂)-1,4-benzoxazepin-5-one [Example 7, Step 6] (300 mg, 0.89 mmol, 1.00 equiv) in Toluene/iPrOH/H₂O (2:1:1, 8 mL) was added K₂CO₃ (494 mg, 3.57 mmol, 4.00 equiv) and [4-(trifluoromethoxy)phenyl]boronic acid (277 mg, 1.35 mmol, 1.50 equiv). The mixture was stirred for 10 min at room temperature. Then Pd(dppf)Cl₂ (16 mg, 0.02 equiv) was added to the solution. The mixture was stirred at 85° C. for 2 h. After cooling the reaction mixture, ethyl acetate (30 mL) was added, and the organic layer was separated. The organic layer was washed with water, dried over anhydrous sodium sulfate, and concentrated under vacuum. The crude product was purified by Prep-HPLC with the following conditions: Column,)(Bridge Prep C18 OBD Column, 5 um, 19*150 mm; mobile phase, Water (10 mmol/L NH₄HCO₃) and CH₃CN (40.0% CH₃CN up to 62.0% in 7 min); Detector, UV 254, 220 nm to afford 180 mg (48%) of 4-(pyrimidin-2-ylmethyl)-7-[4-(trifluoromethoxy)phenyl]-2,3,4,5-tetrahydro(8,9-d₂)-1,4-benzoxazepin-5-one as a white solid. LC-MS: m/z=418 [M+H]⁺

¹H NMR (400 MHz, Chloroform-d) δ 8.73-8.71 (m, 2H), 8.18 (s, 0.6H), 7.62-7.59 (m, 2H), 7.27-7.25 (m, 1H), 7.23-7.21 (m, 1H), 7.20-7.10 (m, 1H), 5.10 (s, 2H), 4.58-4.55 (m, 2H), 3.79-3.76 (m, 2H).

The following compounds in Table 1 can generally be made using the methods described above:

TABLE 1

| No. | Structure |
|---|---|
| 8 | 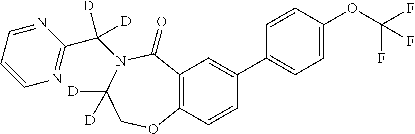 [SMILES: [2H]C1([2H])COc2ccc(-c3ccc(OC(F)(F)F)cc3)cc2C(=O)N1C([2H])([2H])c1ncccn1] |
| 9 | 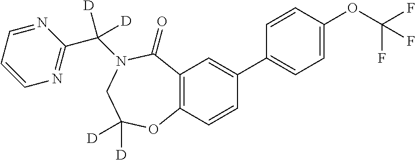 [SMILES: [2H]C1([2H])CN(C([2H])([2H])c2ncccn2)C(=O)c2cc(-c3ccc(OC(F)(F)F)cc3)ccc2O1] |
| 10 | 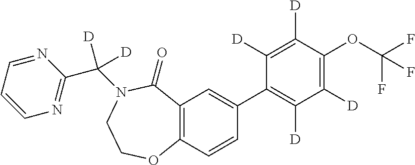 [SMILES: [2H]c1c([2H])c(-c2ccc3c(c2)C(=O)N(C([2H])([2H])c2ncccn2)CCO3)c([2H])c([2H])c1OC(F)(F)F] |
| 11 | 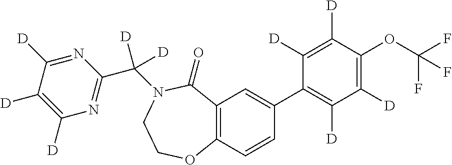 [SMILES: [2H]c1nc(C([2H])([2H])N2CCOc3ccc(-c4c([2H])c([2H])c(OC(F)(F)F)c([2H])c4[2H])cc3C2=O)nc([2H])c1[2H]] |
| 12 | 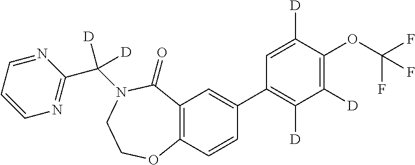 [SMILES: [2H]c1cc(-c2ccc3c(c2)C(=O)N(C([2H])([2H])c2ncccn2)CCO3)c([2H])c([2H])c1OC(F)(F)F] |
| 13 | 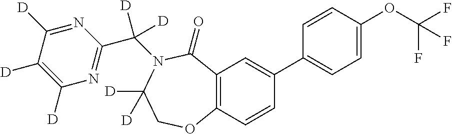 [SMILES: [2H]c1nc(C([2H])([2H])N2C(=O)c3cc(-c4ccc(OC(F)(F)F)cc4)ccc3OCC2([2H])[2H])nc([2H])c1[2H]] |
| 14 | 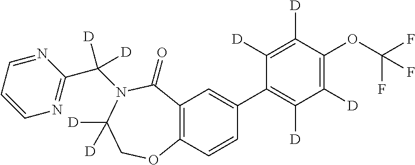 [SMILES: [2H]c1c([2H])c(-c2ccc3c(c2)C(=O)N(C([2H])([2H])c2ncccn2)C([2H])([2H])CO3)c([2H])c([2H])c1OC(F)(F)F] |
| 15 | 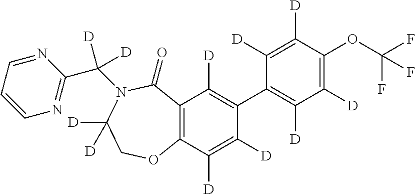 [SMILES: [2H]c1c([2H])c(-c2c([2H])c([2H])c3c(c2[2H])C(=O)N(C([2H])([2H])c2ncccn2)C([2H])([2H])CO3)c([2H])c([2H])c1OC(F)(F)F] |
| 16 | 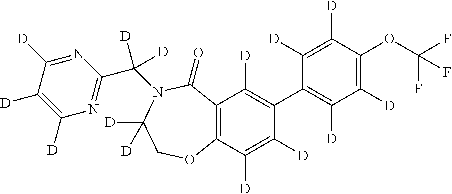 [SMILES: [2H]c1nc(C([2H])([2H])N2C(=O)c3c([2H])c(-c4c([2H])c([2H])c(OC(F)(F)F)c([2H])c4[2H])c([2H])c([2H])c3OCC2([2H])[2H])nc([2H])c1[2H]] |
| 17 | 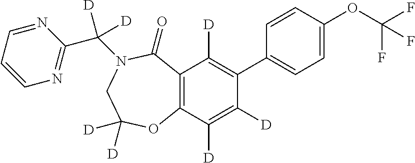 [SMILES: [2H]c1c([2H])c(-c2ccc(OC(F)(F)F)cc2)c([2H])c2c1OC([2H])([2H])CN(C([2H])([2H])c1ncccn1)C2=O] |
| 18 | 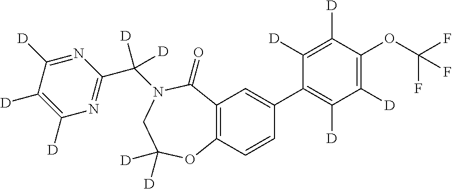 [SMILES: [2H]c1nc(C([2H])([2H])N2CC([2H])([2H])Oc3ccc(-c4c([2H])c([2H])c(OC(F)(F)F)c([2H])c4[2H])cc3C2=O)nc([2H])c1[2H]] |
| 19 | 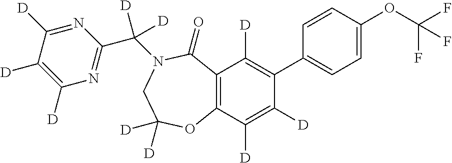 [SMILES: [2H]c1nc(C([2H])([2H])N2CC([2H])([2H])Oc3c([2H])c([2H])c(-c4ccc(OC(F)(F)F)cc4)c([2H])c3C2=O)nc([2H])c1[2H]] |
| 20 | 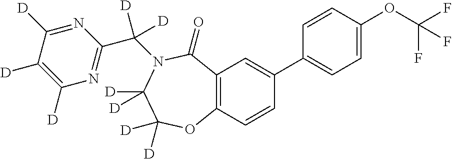 [SMILES: [2H]c1nc(C([2H])([2H])N2C(=O)c3cc(-c4ccc(OC(F)(F)F)cc4)ccc3OC([2H])([2H])C2([2H])[2H])nc([2H])c1[2H]] |
| 21 | 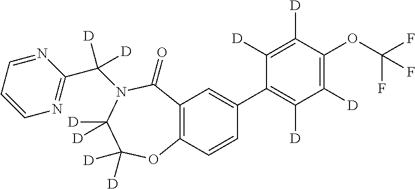 [SMILES: [2H]c1c([2H])c(-c2ccc3c(c2)C(=O)N(C([2H])([2H])c2ncccn2)C([2H])([2H])C([2H])([2H])O3)c([2H])c([2H])c1OC(F)(F)F] |
| 22 | 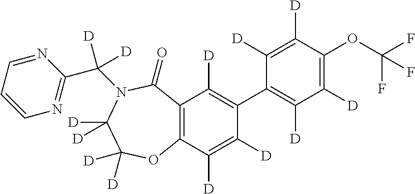 [SMILES: [2H]c1c([2H])c(-c2c([2H])c([2H])c3c(c2[2H])C(=O)N(C([2H])([2H])c2ncccn2)C([2H])([2H])C([2H])([2H])O3)c([2H])c([2H])c1OC(F)(F)F] |
| 23 | 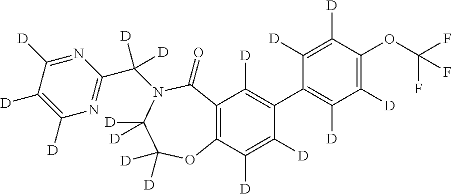 [SMILES: [2H]c1nc(C([2H])([2H])N2C(=O)c3c([2H])c(-c4c([2H])c([2H])c(OC(F)(F)F)c([2H])c4[2H])c([2H])c([2H])c3OC([2H])([2H])C2([2H])[2H])nc([2H])c1[2H]] |
| 24 | 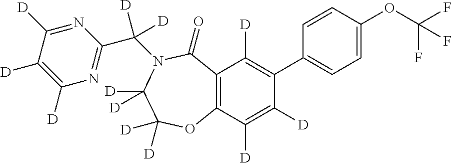 [SMILES: [2H]c1nc(C([2H])([2H])N2C(=O)c3c([2H])c(-c4ccc(OC(F)(F)F)cc4)c([2H])c([2H])c3OC([2H])([2H])C2([2H])[2H])nc([2H])c1[2H]] |
| 25 | 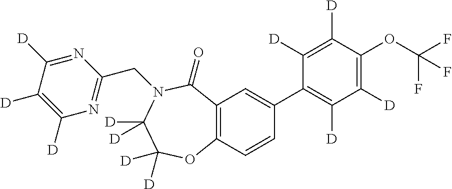 [SMILES: [2H]c1nc(CN2C(=O)c3cc(-c4c([2H])c([2H])c(OC(F)(F)F)c([2H])c4[2H])ccc3OC([2H])([2H])C2([2H])[2H])nc([2H])c1[2H]] |
| 26 | 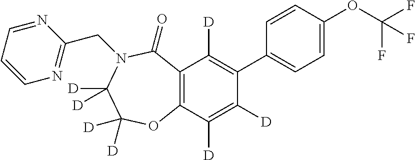 [SMILES: [2H]c1c([2H])c(-c2ccc(OC(F)(F)F)cc2)c([2H])c2c1OC([2H])([2H])C([2H])([2H])N(Cc1ncccn1)C2=O] |
| 27 | 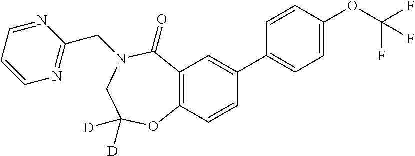 [SMILES: [2H]C1([2H])CN(Cc2ncccn2)C(=O)c2cc(-c3ccc(OC(F)(F)F)cc3)ccc2O1] |
| 28 | 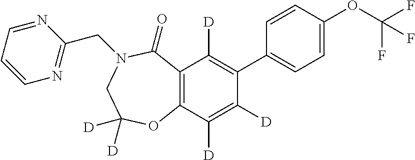 [SMILES: [2H]c1c([2H])c(-c2ccc(OC(F)(F)F)cc2)c([2H])c2c1OC([2H])([2H])CN(Cc1ncccn1)C2=O] |
| 29 | 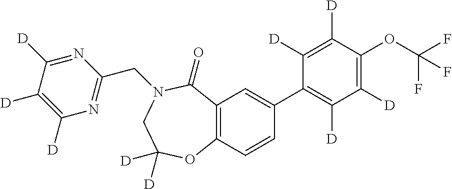 [SMILES: [2H]c1nc(CN2CC([2H])([2H])Oc3ccc(-c4c([2H])c([2H])c(OC(F)(F)F)c([2H])c4[2H])cc3C2=O)nc([2H])c1[2H]] |
| 30 | 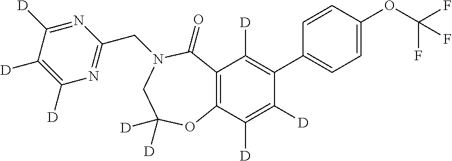 [SMILES: [2H]c1nc(CN2CC([2H])([2H])Oc3c([2H])c([2H])c(-c4ccc(OC(F)(F)F)cc4)c([2H])c3C2=O)nc([2H])c1[2H]] |
| 31 | 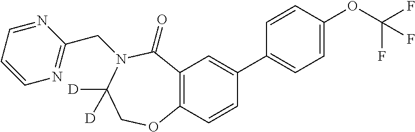 [SMILES: [2H]C1([2H])COc2ccc(-c3ccc(OC(F)(F)F)cc3)cc2C(=O)N1Cc1ncccn1] |
| 32 | 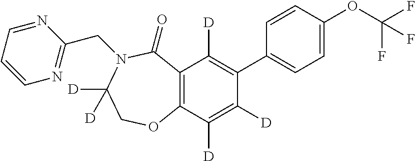 [SMILES: [2H]c1c([2H])c(-c2ccc(OC(F)(F)F)cc2)c([2H])c2c1OCC([2H])([2H])N(Cc1ncccn1)C2=O] |
| 33 | 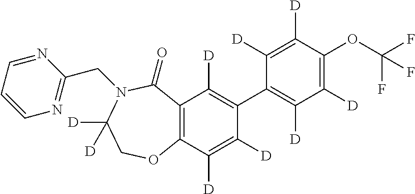 [SMILES: [2H]c1c([2H])c(-c2c([2H])c([2H])c3c(c2[2H])C(=O)N(Cc2ncccn2)C([2H])([2H])CO3)c([2H])c([2H])c1OC(F)(F)F] |
| 34 | 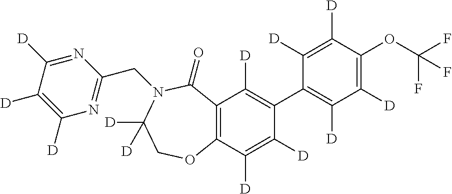 [SMILES: [2H]c1nc(CN2C(=O)c3c([2H])c(-c4c([2H])c([2H])c(OC(F)(F)F)c([2H])c4[2H])c([2H])c([2H])c3OCC2([2H])[2H])nc([2H])c1[2H]] |
| 35 | 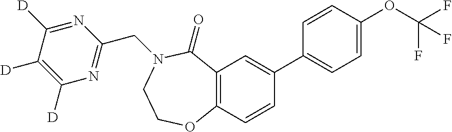 [SMILES: [2H]c1nc(CN2CCOc3ccc(-c4ccc(OC(F)(F)F)cc4)cc3C2=O)nc([2H])c1[2H]] |
| 36 | 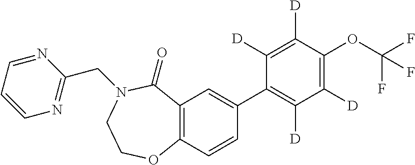 [SMILES: [2H]c1c([2H])c(-c2ccc3c(c2)C(=O)N(Cc2ncccn2)CCO3)c([2H])c([2H])c1OC(F)(F)F] |
| 37 | 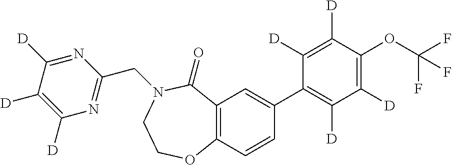 [SMILES: [2H]c1nc(CN2CCOc3ccc(-c4c([2H])c([2H])c(OC(F)(F)F)c([2H])c4[2H])cc3C2=O)nc([2H])c1[2H]] |
| 38 | 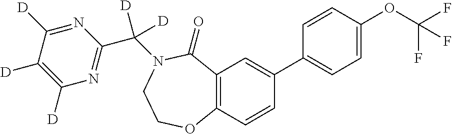 [SMILES: [2H]c1nc(C([2H])([2H])N2CCOc3ccc(-c4ccc(OC(F)(F)F)cc4)cc3C2=O)nc([2H])c1[2H]] |
| 39 | 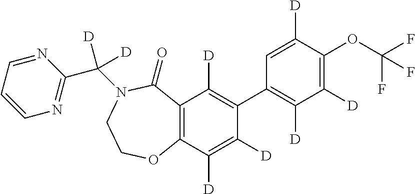 [SMILES: [2H]c1cc(-c2c([2H])c([2H])c3c(c2[2H])C(=O)N(C([2H])([2H])c2ncccn2)CCO3)c([2H])c([2H])c1OC(F)(F)F] |
| 40 | 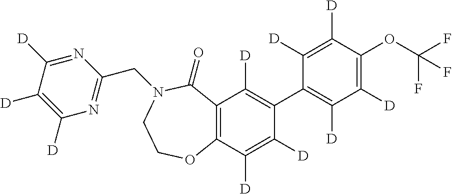 [SMILES: [2H]c1nc(CN2CCOc3c([2H])c([2H])c(-c4c([2H])c([2H])c(OC(F)(F)F)c([2H])c4[2H])c([2H])c3C2=O)nc([2H])c1[2H]] |
| 41 | 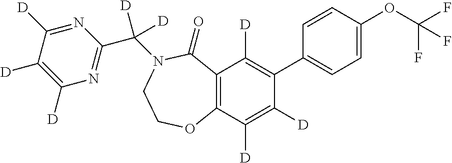 [SMILES: [2H]c1nc(C([2H])([2H])N2CCOc3c([2H])c([2H])c(-c4ccc(OC(F)(F)F)cc4)c([2H])c3C2=O)nc([2H])c1[2H]] |
| 42 | 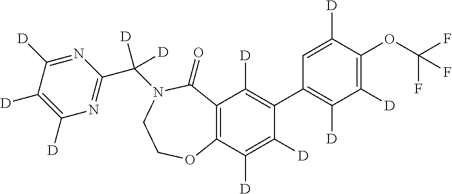 [SMILES: [2H]c1cc(-c2c([2H])c([2H])c3c(c2[2H])C(=O)N(C([2H])([2H])c2nc([2H])c([2H])c([2H])n2)CCO3)c([2H])c([2H])c1OC(F)(F)F] |
| 43 | 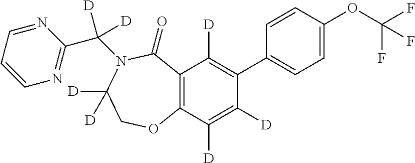 [SMILES: [2H]c1c([2H])c(-c2ccc(OC(F)(F)F)cc2)c([2H])c2c1OCC([2H])([2H])N(C([2H])([2H])c1ncccn1)C2=O] |
| 44 | 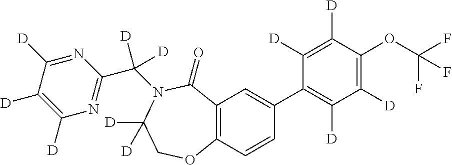 [SMILES: [2H]c1nc(C([2H])([2H])N2C(=O)c3cc(-c4c([2H])c([2H])c(OC(F)(F)F)c([2H])c4[2H])ccc3OCC2([2H])[2H])nc([2H])c1[2H]] |
| 45 | 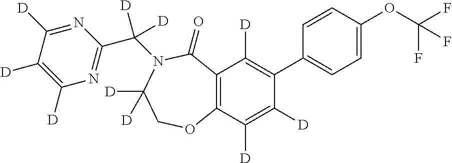 [SMILES: [2H]c1nc(C([2H])([2H])N2C(=O)c3c([2H])c(-c4ccc(OC(F)(F)F)cc4)c([2H])c([2H])c3OCC2([2H])[2H])nc([2H])c1[2H]] |
| 46 | 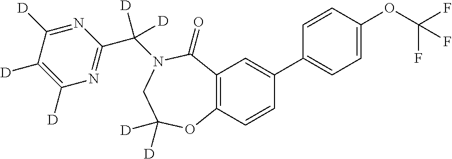 [SMILES: [2H]c1nc(C([2H])([2H])N2CC([2H])([2H])Oc3ccc(-c4ccc(OC(F)(F)F)cc4)cc3C2=O)nc([2H])c1[2H]] |
| 47 | 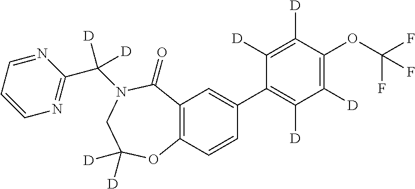 [SMILES: [2H]c1c([2H])c(-c2ccc3c(c2)C(=O)N(C([2H])([2H])c2ncccn2)CC([2H])([2H])O3)c([2H])c([2H])c1OC(F)(F)F] |
| 48 | 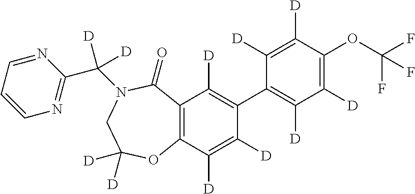 [SMILES: [2H]c1c([2H])c(-c2c([2H])c([2H])c3c(c2[2H])C(=O)N(C([2H])([2H])c2ncccn2)CC([2H])([2H])O3)c([2H])c([2H])c1OC(F)(F)F] |
| 49 | 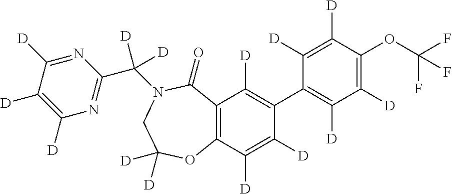 [SMILES: [2H]c1nc(C([2H])([2H])N2CC([2H])([2H])Oc3c([2H])c([2H])c(-c4c([2H])c([2H])c(OC(F)(F)F)c([2H])c4[2H])c([2H])c3C2=O)nc([2H])c1[2H]] |
| 50 | 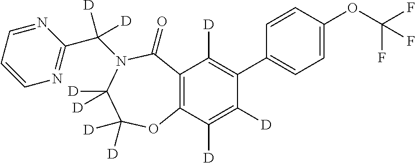 [SMILES: [2H]c1c([2H])c(-c2ccc(OC(F)(F)F)cc2)c([2H])c2c1OC([2H])([2H])C([2H])([2H])N(C([2H])([2H])c1ncccn1)C2=O] |
| 51 | 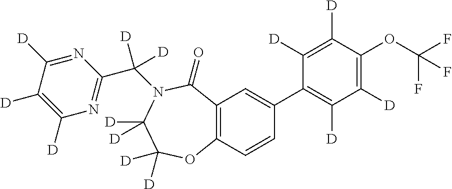 [SMILES: [2H]c1nc(C([2H])([2H])N2C(=O)c3cc(-c4c([2H])c([2H])c(OC(F)(F)F)c([2H])c4[2H])ccc3OC([2H])([2H])C2([2H])[2H])nc([2H])c1[2H]] |
| 52 | 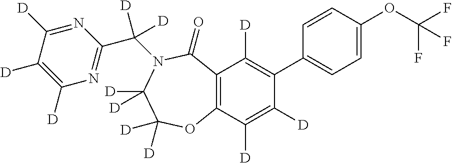 [SMILES: [2H]c1nc(C([2H])([2H])N2C(=O)c3c([2H])c(-c4ccc(OC(F)(F)F)cc4)c([2H])c([2H])c3OC([2H])([2H])C2([2H])[2H])nc([2H])c1[2H]] |
| 53 | 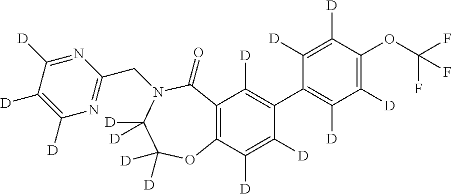 [SMILES: [2H]c1nc(CN2C(=O)c3c([2H])c(-c4c([2H])c([2H])c(OC(F)(F)F)c([2H])c4[2H])c([2H])c([2H])c3OC([2H])([2H])C2([2H])[2H])nc([2H])c1[2H]] |
| 54 | 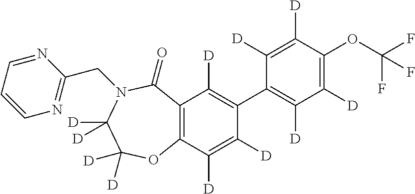 [SMILES: [2H]c1c([2H])c(-c2c([2H])c([2H])c3c(c2[2H])C(=O)N(Cc2ncccn2)C([2H])([2H])C([2H])([2H])O3)c([2H])c([2H])c1OC(F)(F)F] |
| 55 | 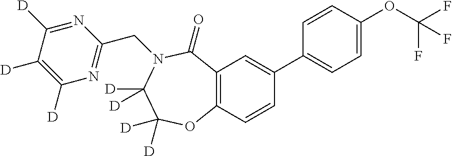 [SMILES: [2H]c1nc(CN2C(=O)c3cc(-c4ccc(OC(F)(F)F)cc4)ccc3OC([2H])([2H])C2([2H])[2H])nc([2H])c1[2H]] |
| 56 | 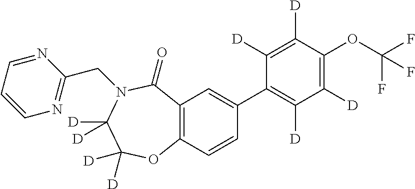 [SMILES: [2H]c1c([2H])c(-c2ccc3c(c2)C(=O)N(Cc2ncccn2)C([2H])([2H])C([2H])([2H])O3)c([2H])c([2H])c1OC(F)(F)F] |
| 57 | 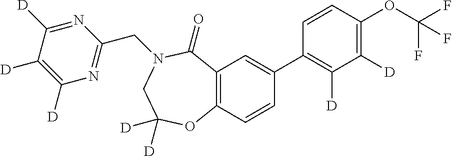 [SMILES: [2H]c1nc(CN2CC([2H])([2H])Oc3ccc(-c4ccc(OC(F)(F)F)c([2H])c4[2H])cc3C2=O)nc([2H])c1[2H]] |
| 58 | 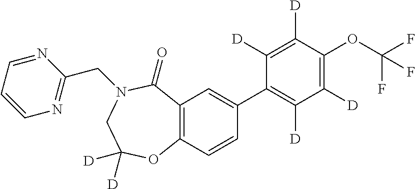 [SMILES: [2H]c1c([2H])c(-c2ccc3c(c2)C(=O)N(Cc2ncccn2)CC([2H])([2H])O3)c([2H])c([2H])c1OC(F)(F)F] |
| 59 | 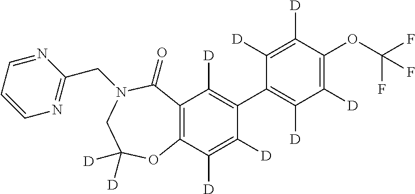 [SMILES: [2H]c1c([2H])c(-c2c([2H])c([2H])c3c(c2[2H])C(=O)N(Cc2ncccn2)CC([2H])([2H])O3)c([2H])c([2H])c1OC(F)(F)F] |
| 60 | 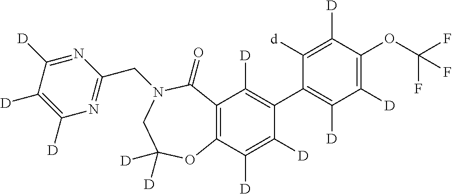 |
| 61 | 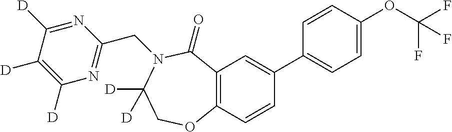 [SMILES: [2H]c1nc(CN2C(=O)c3cc(-c4ccc(OC(F)(F)F)cc4)ccc3OCC2([2H])[2H])nc([2H])c1[2H]] |
| 62 | 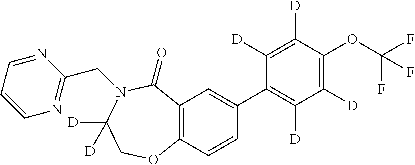 [SMILES: [2H]c1c([2H])c(-c2ccc3c(c2)C(=O)N(Cc2ncccn2)C([2H])([2H])CO3)c([2H])c([2H])c1OC(F)(F)F] |
| 63 | 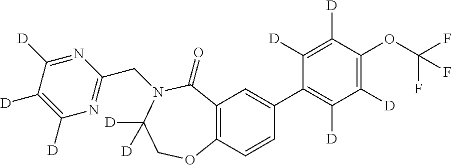 [SMILES: [2H]c1nc(CN2C(=O)c3cc(-c4c([2H])c([2H])c(OC(F)(F)F)c([2H])c4[2H])ccc3OCC2([2H])[2H])nc([2H])c1[2H]] |
| 64 | 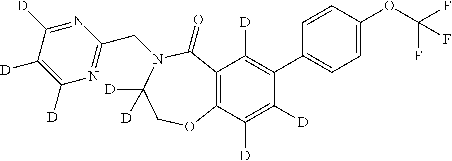 [SMILES: [2H]c1nc(CN2C(=O)c3c([2H])c(-c4ccc(OC(F)(F)F)cc4)c([2H])c([2H])c3OCC2([2H])[2H])nc([2H])c1[2H]] |
| 65 | 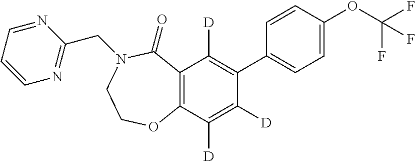 [SMILES: [2H]c1c([2H])c(-c2ccc(OC(F)(F)F)cc2)c([2H])c2c1OCCN(Cc1ncccn1)C2=O] |
| 66 | 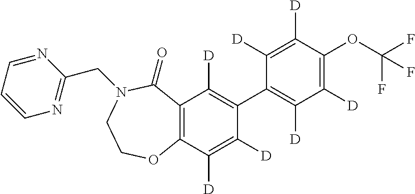 [SMILES: [2H]c1c([2H])c(-c2c([2H])c([2H])c3c(c2[2H])C(=O)N(Cc2ncccn2)CCO3)c([2H])c([2H])c1OC(F)(F)F] |
| 67 | 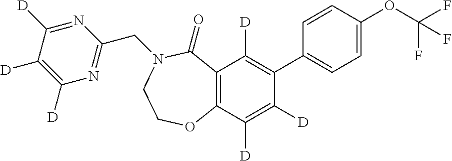 [SMILES: [2H]c1nc(CN2CCOc3c([2H])c([2H])c(-c4ccc(OC(F)(F)F)cc4)c([2H])c3C2=O)nc([2H])c1[2H]] |

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

Biological Activity Assays

In Vitro Liver Microsomal Stability Assay

Human liver microsomal stability assays were conducted at 0.5 mg per mL liver microsome protein with an NADPH-generating system consisting of NADP (1 mM, pH 7.4), glucose-5-phosphate (5 mM, pH 7.4), and glucose-6-phosphate dehydrogenase (1 unit/mL).

Test compounds were prepared as solutions in DMSO and added to the assay mixture (1 μM, final concentration in incubation) to be incubated at 37±1° C. Reactions were initiated with the addition of cofactor and were stopped at 0, 15, 30, 45, and 60 min after cofactor addition with stop reagent (0.2 mL acetonitrile). Samples were centrifuged (920×g for 10 min at 10° C.) in 96-well plates. Supernatant fractions were analyzed by LC-MS/MS to determine the percent remaining and estimate the degradation half-life of the test compounds. The results are presented in Table 2 below.

TABLE 2

| | Clearance % | Half-Life % |
|---|---|---|
| Example # | (mL/min/kg) | (min) |
| 1 | 12.29 | 141.43 |
| 2 | 3.04 | 571.12 |
| 3 | 6.27 | 277.20 |
| 4 | 14.39 | 120.77 |
| 5 | 21.49 | 80.90 |
| 6 | 19.17 | 90.68 |
| 7 | 12.49 | 139.22 |

In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

Monoamine Oxidase a Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

Monooamine Oxidase B Inhibition and Oxidative Turnover

The procedure is carried out as described in Uebelhack, Pharmacopsychiatry 1998, 31(5), 187-192, which is hereby incorporated by reference in its entirety.

In Vitro Late I_{Na} Screening Assay

The procedure can be carried out as described in U.S. Pat. No. 8,586,732, which is herein incorporated by reference in its entirety.

In Vitro Peak I_{Na} Screening Assay

The procedure can be carried out as described in U.S. Pat. No. 8,586,732, which is herein incorporated by reference in its entirety.

In Vitro hERG Screening Assay

The procedure can be carried out as described in U.S. Pat. No. 8,586,732, which is herein incorporated by reference in its entirety.

In Vivo I_{Na} Assay

The procedure can be carried out as described in U.S. Pat. No. 8,586,732, which is herein incorporated by reference in its entirety.

From the foregoing description, one skilled in the art can ascertain the essential characteristics of this invention, and without departing from the spirit and scope thereof, can make various changes and modifications of the invention to adapt it to various usages and conditions.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A compound of Formula (I): 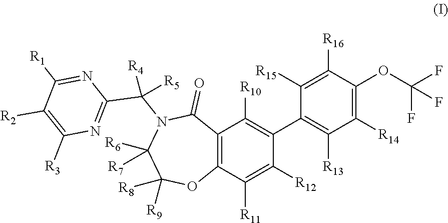
or a pharmaceutically acceptable salt, ester, prodrug, or solvate thereof, wherein:
R₁-R₁₆ are, independently, hydrogen or deuterium;
at least one of R₁-R₁₆ is deuterium;
with the proviso that when all of R₆, R₇, R₈, and R₉ are deuterium, at least one of R₁-R₅ or R₁₀-R₁₆ are also deuterium; and
at least one of R₁-R₁₆ has deuterium enrichment of at least about 1%.

2. The compound of claim 1, wherein R₄ and R₅ are deuterium.

3. The compound of claim 1, wherein R₄-R₅ and R₆-R₇ are deuterium.

4. The compound of claim 1, wherein R₄-R₅ and R₈-R₉ are deuterium.

5. The compound of claim 1, wherein R₄-R₅, R₆-R₇, and R₈-R₉ are deuterium.

6. The compound of claim 1, wherein R₁-R₃ are deuterium.

7. The compound of claim 1, wherein R₁₀-R₁₂ are deuterium.

8. The compound of claim 1, wherein R₁₃-R₁₆ are deuterium.

9. The compound of claim 1, wherein R₆ and R₇ are hydrogen.

10. The compound of claim 1, wherein R₅ and R₉ are hydrogen.

11. The compound of claim 1, wherein R₆-R₉ are hydrogen.

12. The compound of claim 1, wherein the compound is:

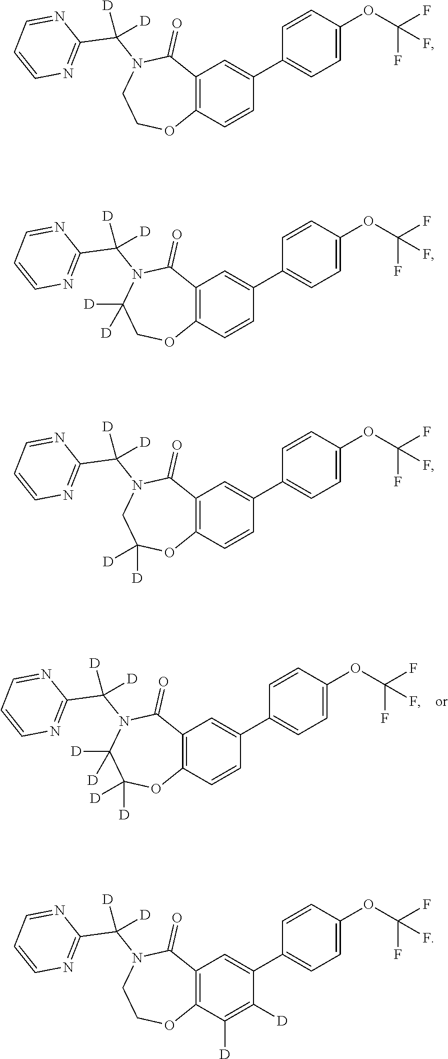 [SMILES: [2H]C([2H])(c1ncccn1)N1CCOc2ccc(-c3ccc(OC(F)(F)F)cc3)cc2C1=O ; [2H]C1([2H])COc2ccc(-c3ccc(OC(F)(F)F)cc3)cc2C(=O)N1C([2H])([2H])c1ncccn1 ; [2H]C1([2H])CN(C([2H])([2H])c2ncccn2)C(=O)c2cc(-c3ccc(OC(F)(F)F)cc3)ccc2O1 ; [2H]C([2H])(c1ncccn1)N1C(=O)c2cc(-c3ccc(OC(F)(F)F)cc3)ccc2OC([2H])([2H])C1([2H])[2H] ; [2H]c1c(-c2ccc(OC(F)(F)F)cc2)cc2c(c1[2H])OCCN(C([2H])([2H])c1ncccn1)C2=O]

13. The compound of claim 1, wherein the compound is:

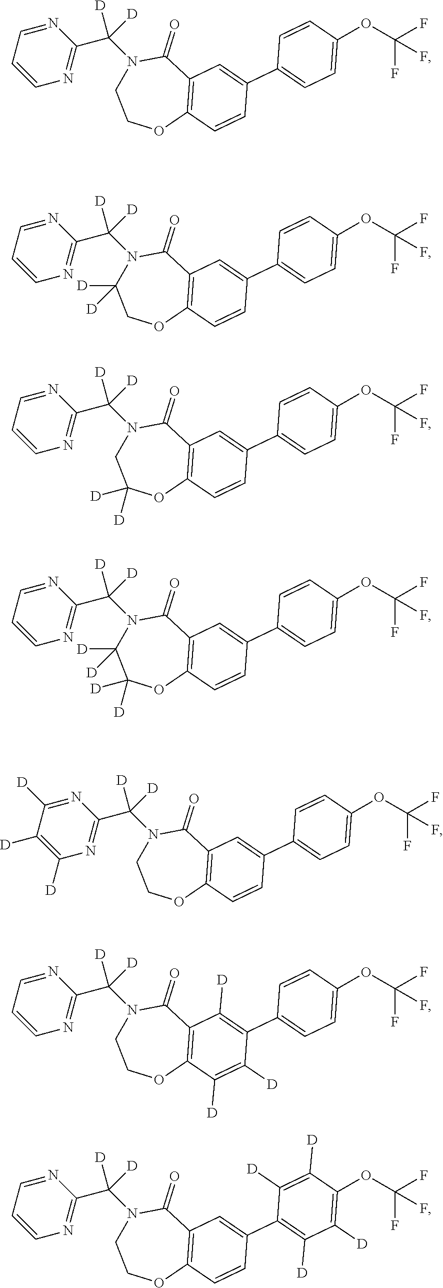

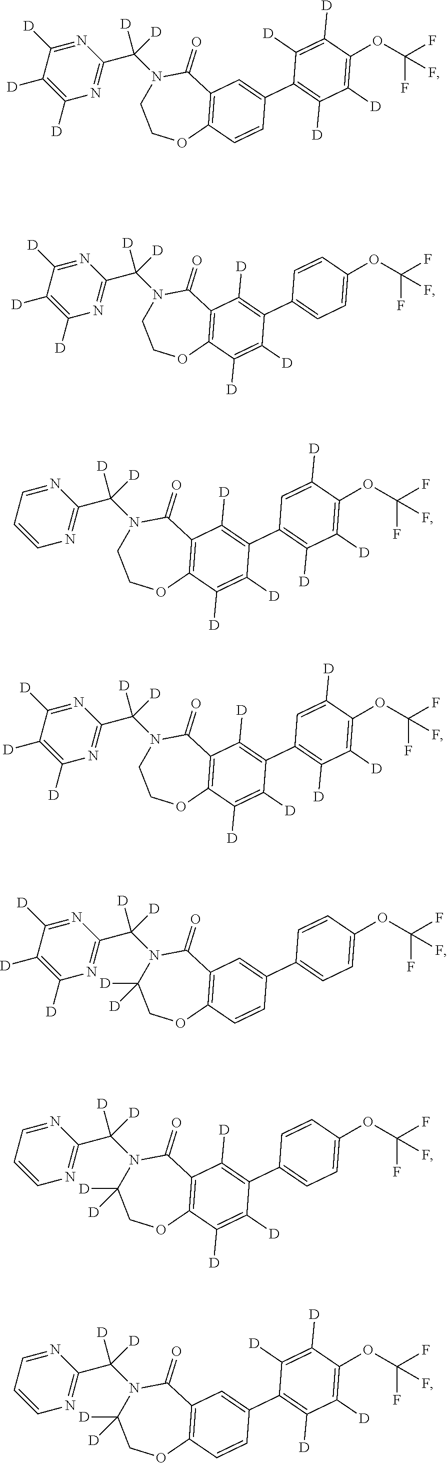

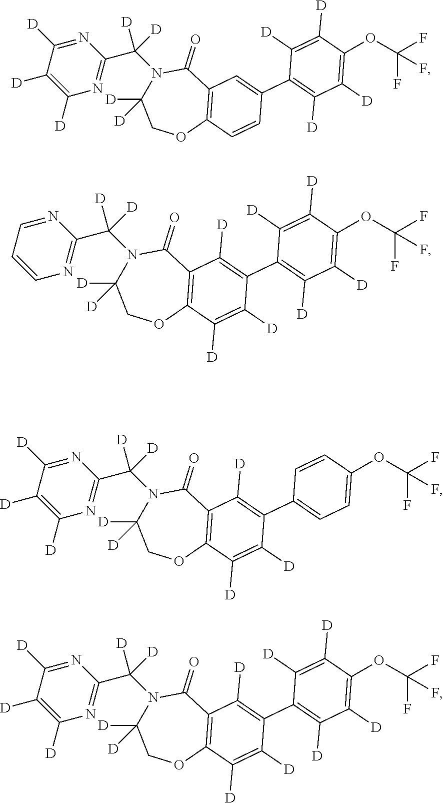 [SMILES: [2H]c1nc(C([2H])([2H])N2C(=O)c3cc(-c4c([2H])c([2H])c(OC(F)(F)F)c([2H])c4[2H])ccc3OCC2([2H])[2H])nc([2H])c1[2H] ; [2H]c1c([2H])c(-c2c([2H])c([2H])c3c(c2[2H])C(=O)N(C([2H])([2H])c2ncccn2)C([2H])([2H])CO3)c([2H])c([2H])c1OC(F)(F)F ; [2H]c1nc(C([2H])([2H])N2C(=O)c3c([2H])c(-c4ccc(OC(F)(F)F)cc4)c([2H])c([2H])c3OCC2([2H])[2H])nc([2H])c1[2H] ; [2H]c1nc(C([2H])([2H])N2C(=O)c3c([2H])c(-c4c([2H])c([2H])c(OC(F)(F)F)c([2H])c4[2H])c([2H])c([2H])c3OCC2([2H])[2H])nc([2H])c1[2H]]

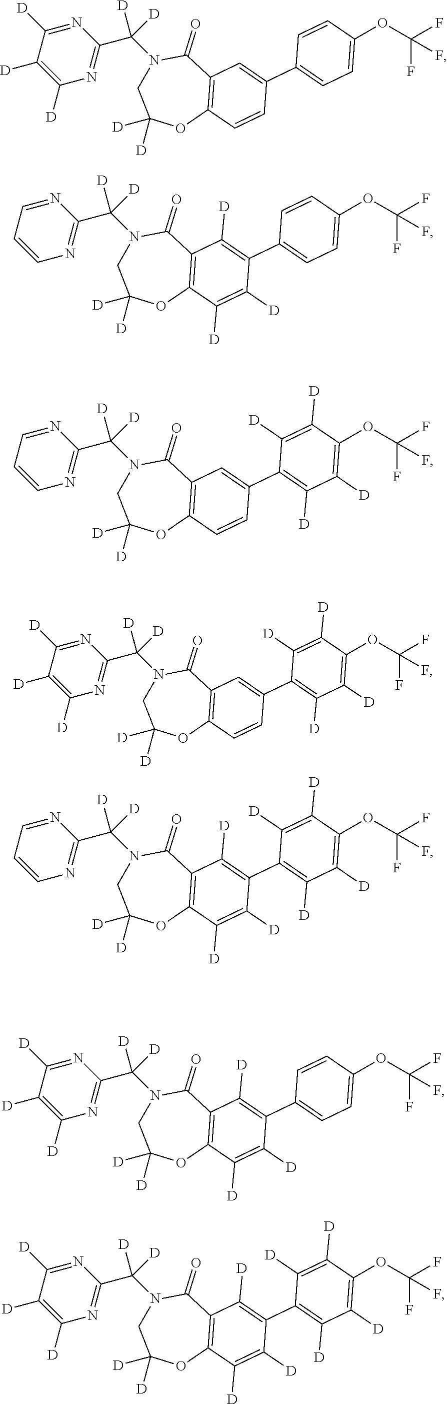

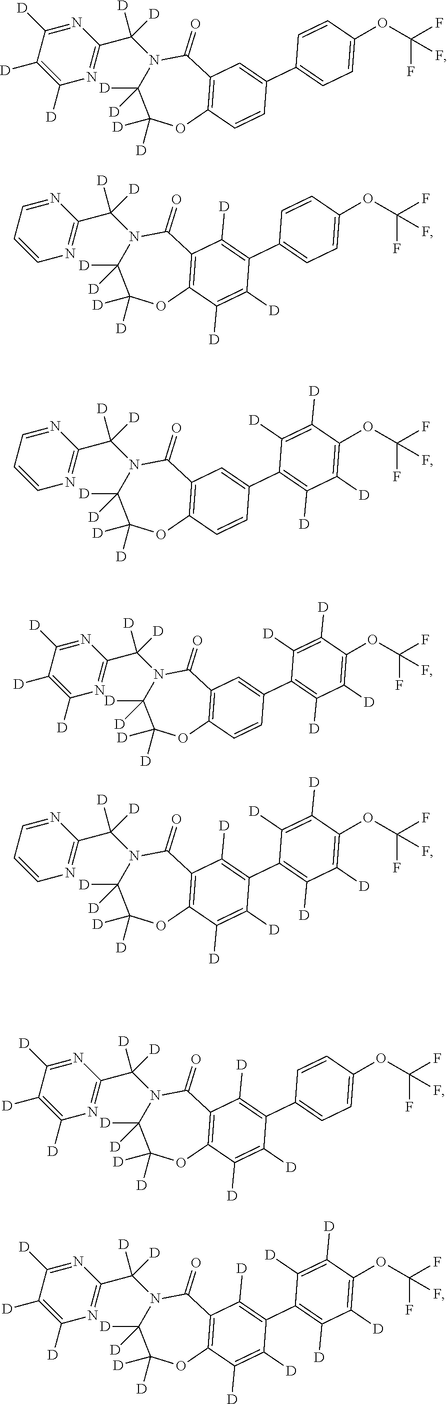

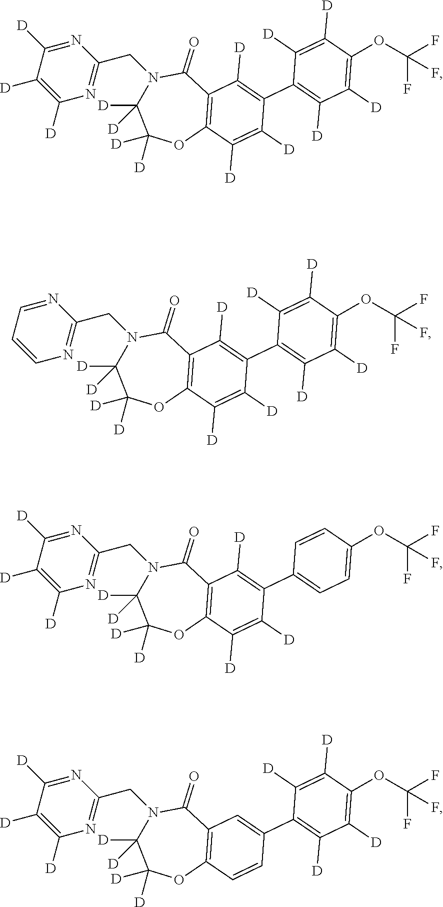 [SMILES: [2H]c1nc(CN2C(=O)c3c([2H])c(-c4c([2H])c([2H])c(OC(F)(F)F)c([2H])c4[2H])c([2H])c([2H])c3OC([2H])([2H])C2([2H])[2H])nc([2H])c1[2H] ; [2H]c1c([2H])c(-c2c([2H])c([2H])c3c(c2[2H])C(=O)N(Cc2ncccn2)C([2H])([2H])C([2H])([2H])O3)c([2H])c([2H])c1OC(F)(F)F ; [2H]c1nc(CN2C(=O)c3c([2H])c(-c4ccc(OC(F)(F)F)cc4)c([2H])c([2H])c3OC([2H])([2H])C2([2H])[2H])nc([2H])c1[2H] ; [2H]c1nc(CN2C(=O)c3cc(-c4c([2H])c([2H])c(OC(F)(F)F)c([2H])c4[2H])ccc3OC([2H])([2H])C2([2H])[2H])nc([2H])c1[2H]]

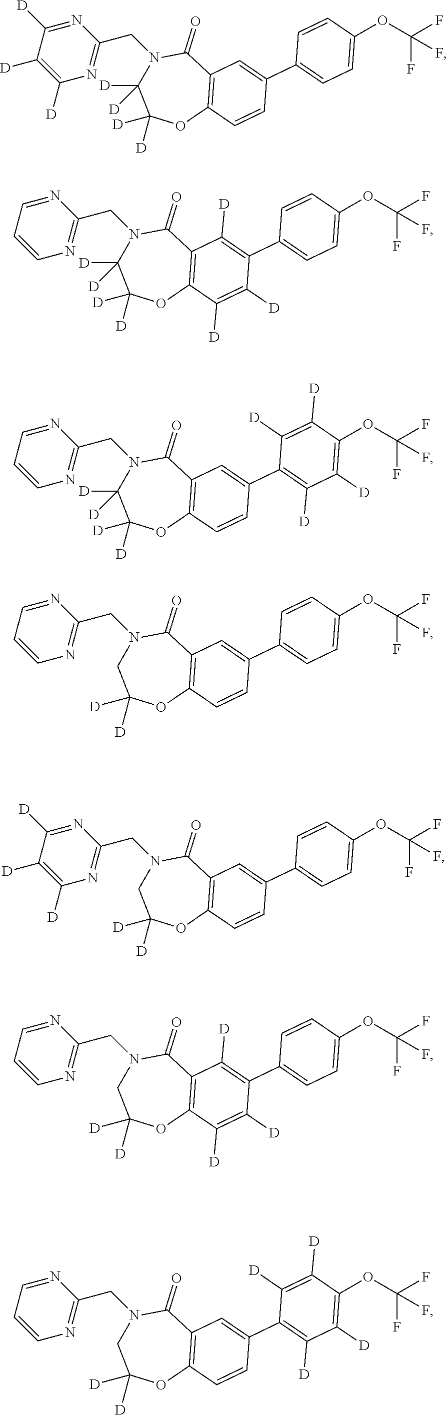

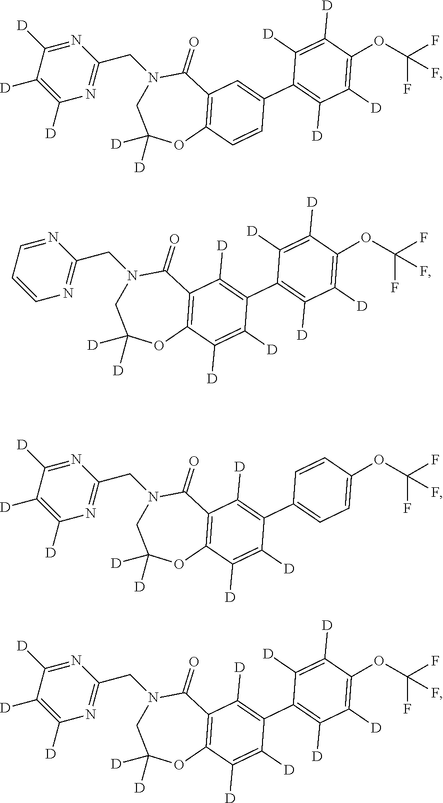 [SMILES: [2H]c1nc(CN2CC([2H])([2H])Oc3ccc(-c4c([2H])c([2H])c(OC(F)(F)F)c([2H])c4[2H])cc3C2=O)nc([2H])c1[2H] ; [2H]c1c([2H])c(-c2c([2H])c([2H])c3c(c2[2H])C(=O)N(Cc2ncccn2)CC([2H])([2H])O3)c([2H])c([2H])c1OC(F)(F)F ; [2H]c1nc(CN2CC([2H])([2H])Oc3c([2H])c([2H])c(-c4ccc(OC(F)(F)F)cc4)c([2H])c3C2=O)nc([2H])c1[2H] ; [2H]c1nc(CN2CC([2H])([2H])Oc3c([2H])c([2H])c(-c4c([2H])c([2H])c(OC(F)(F)F)c([2H])c4[2H])c([2H])c3C2=O)nc([2H])c1[2H]]

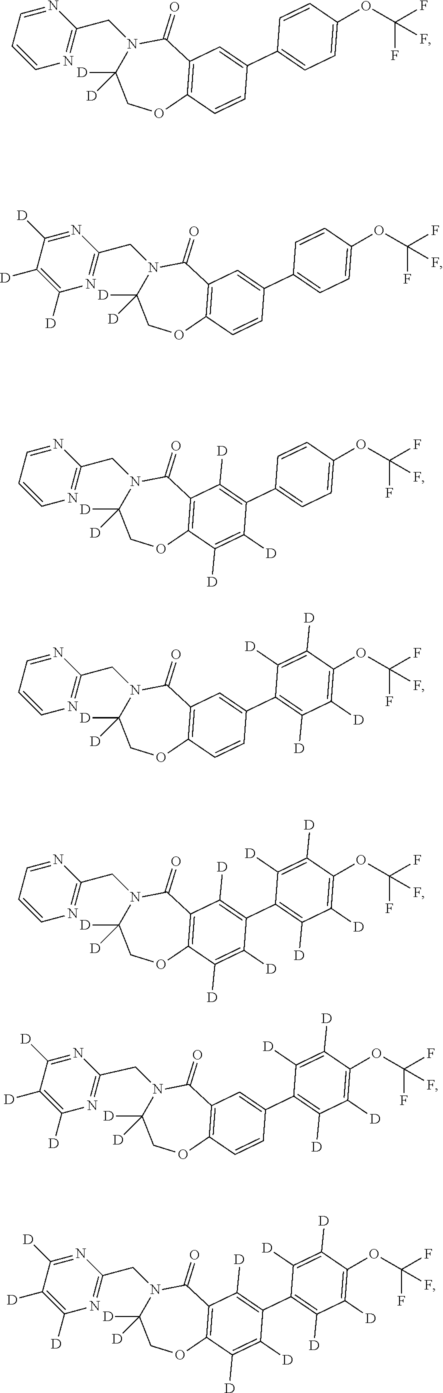

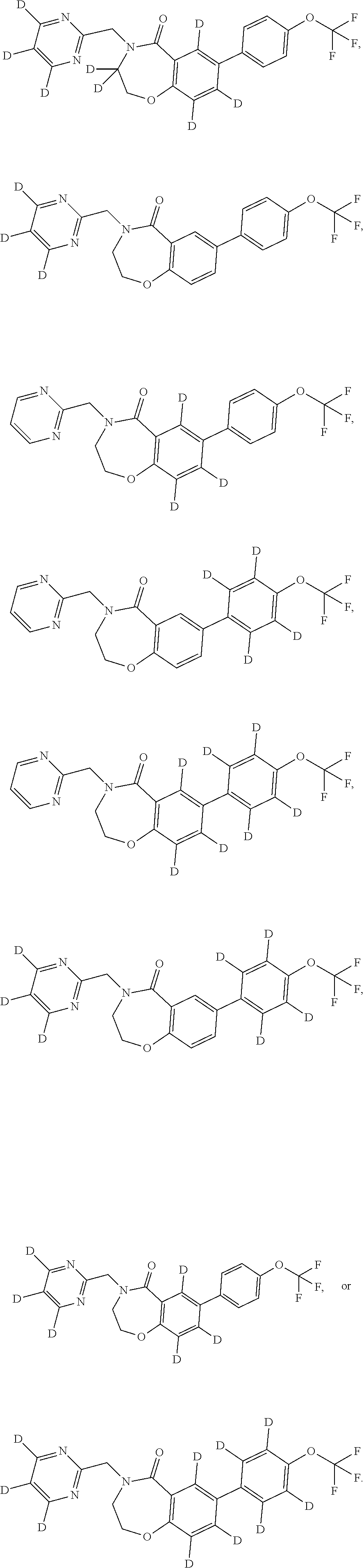

14. The compound of claim 1, wherein the compound is a pharmaceutically acceptable salt that is a hydrochloride, a hydrobromide, a sulfate, a formate, an acetate, a fumarate, a citrate, a tartrate, a mesylate, a tosylate, or a besylate.

15. The compound of claim 1, wherein the compound has deuterium enrichment of at least about 10% in at least one of the positions R₁-R₁₆ in the compound of Formula I.

16. A pharmaceutical composition comprising the compound of claim 1 and a pharmaceutically acceptable carrier.

17. A method of treating or preventing a late sodium current-mediated disorder, the method comprising administering, to a patient in need thereof, a therapeutically effective amount of the compound of claim 1.

18. A method of treating or preventing a late sodium current-mediated disorder, the method comprising administering, to a patient in need thereof, the pharmaceutical composition of claim 16.

19. The method of claim 17, wherein the late sodium current-mediated disorder is acute coronary syndrome, a peripheral arterial disease, intermittent claudication, Prinzmetal's (variant) angina, stable angina, unstable angina, ischemia, recurrent ischemia, reperfusion injury, exercise induced angina, pulmonary hypertension, congestive heart disease, myocardial infarction, cardiomyopathy, hypertrophic cardiomyopathy, heart failure, atrial fibrillation (AF), atrial premature beats (APBs), an ischemic heart disorder, myocardial ischemia, arrhythmia, congestive heart failure, long QT syndrome, diabetes, reduced insulin sensitivity, a disease affecting the neuro-muscular system, an inflammatory disease, diabetic peripheral neuropathy, or a proliferative disease.

20. The method of claim 18, wherein the late sodium current-mediated disorder is selected from acute coronary syndrome, peripheral arterial disease, intermittent claudication, Prinzmetal's (variant) angina, stable angina, unstable angina, ischemia, recurrent ischemia, reperfusion injury, exercise induced angina, pulmonary hypertension, congestive heart disease, myocardial infarction, cardiomyopathy, hypertrophic cardiomyopathy, heart failure, atrial fibrillation (AF), atrial premature beats (APBs), ischemic heart disorders, myocardial ischemia, arrhythmias, congestive heart failure, long QT syndrome, diabetes, reduced insulin sensitivity, diseases affecting the neuro-muscular system, inflammatory diseases, diabetic peripheral neuropathy, and proliferative diseases.

21. The method of claim 17, further comprising administering an additional therapeutic agent in combination with the compound.

22. The method of claim 18, further comprising administering of an additional therapeutic agent in combination with the compound.

23. A compound of Formula Ia: 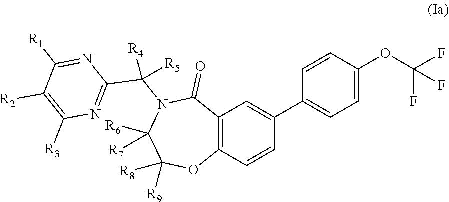
or a pharmaceutically acceptable salt, ester, prodrug, or solvate thereof, wherein:
R₁-R₉ are, independently, hydrogen or deuterium,
at least one of R₁-R₅ is deuterium,
when any of R₆, R₇, R₈, and R₉ are deuterium, at least one of R₁-R₅ are also deuterium; and
at least one of R₁-R₉ has deuterium enrichment of at least about 1%.

24. A compound of Formula (III), (IV), or (V): 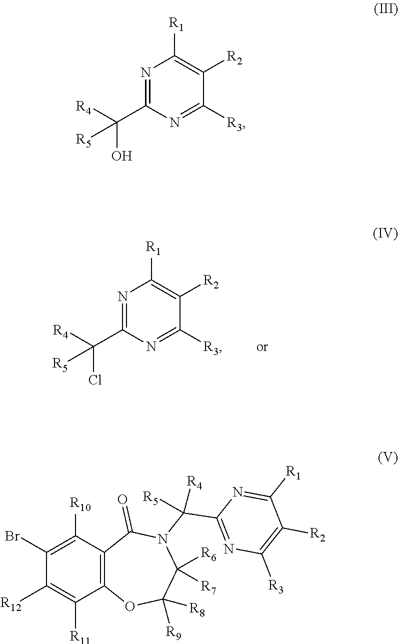
or a pharmaceutically acceptable salt, ester, prodrug, or solvate thereof, wherein:
(i) in the compound of formula (III) or (IV):
R₁-R₅ are, independently, hydrogen or deuterium,
at least one of R₁-R₅ is deuterium, and
at least one of R₁-R₅ has deuterium enrichment of at least about 1%; and
(ii) in the compound of formula (V):
R₁-R₁₂ are, independently, hydrogen or deuterium;
at least one R₁-R₁₂ is deuterium,
at least one of R₁-R₅ or R₁₀-R₁₆ are deuterium when all of R₆, R₇, R₈, and R₉ are deuterium; and
at least one of R₁-R₁₂ has deuterium enrichment of at least about 1%.
